(19) United States
(12) Patent Application Publication
(10) Pub. No.: US 2023/0415157 A1
(43) Pub. Date: Dec. 28, 2023

(54) INTEGRATED MICROFLUIDIC SYSTEM FOR THE PROCESSING OF TISSUES INTO CELLULAR SUSPENSIONS

(71) Applicant: THE REGENTS OF THE UNIVERSITY OF CALIFORNIA, Oakland, CA (US)

(72) Inventors: Jered Haun, Irvine, CA (US); Jeremy A. Lombardo, Irvine, CA (US)

(73) Assignee: THE REGENTS OF THE UNIVERSITY OF CALIFORNIA, Oakland, CA (US)

(21) Appl. No.: 18/030,959

(22) PCT Filed: Oct. 11, 2021
(86) PCT No.: PCT/US2021/054440

§ 371 Date: Apr. 7, 2023

Related U.S. Application Data
(60) Provisional application No. 63/090,497, filed on Oct. 12, 2020.

(51) Int. Cl. B01L 3/00; C12M 3/06; C12M 1/00

(52) U.S. Cl. CPC B01L 3/502753; C12M 23/16; C12M 45/09; B01L 2300/0816; B01L 2300/0864; B01L 2400/0622; C12Y 304/24007; B01L 2300/0681

(57) ABSTRACT

A microfluidic system for processing a tissue sample includes a microfluidic digestion device having an outlet fluidically connected to an inlet of a dissociation/filter device. The microfluidic digestion device includes an inlet and an outlet and a tissue chamber that connects to plurality of upstream fluidic channels and a plurality of downstream fluidic channels. The microfluidic dissociation/filter device includes an inlet, a first outlet, a second outlet, and a plurality of furcating dissociation channels having a plurality of expansion and constriction regions disposed along a length thereof, wherein one or more filters are disposed in a flow path downstream of the plurality of furcating dissociation channels. Pumps are provided to pump buffer and/or enzyme-containing fluid through the digestion device and dissociation/filter device. Tissue is initially processed in the digestion device and then passes into the dissociation/filter device.

### RELATED APPLICATION

This application claims priority to U.S. Provisional Patent Application No. 63/090,497 filed on Oct. 12, 2020, which is hereby incorporated by reference. Priority is claimed pursuant to 35 U.S.C. § 119 and any other applicable statute.

### STATEMENT REGARDING FEDERALLY SPONSORED RESEARCH AND DEVELOPMENT

This invention was made with Government support under Grant No. IIP-1362165, awarded by the National Science Foundation (NSF). The Government has certain rights in the invention.

### TECHNICAL FIELD

The technical field relates to microfluidic devices that are used to process tissue specimens or tissue samples into cellular suspensions.

### BACKGROUND

Tissues are highly complex ecosystems containing a diverse array of cell subtypes. Significant variation can also arise within a given subtype due to differences in activation state, genetic mutations, epigenetic distinctions, stochastic events, and microenvironmental factors. This has led to a rapid growth in studies attempting to capture cellular heterogeneity, and thereby gain a better understanding of tissue and organ development, normal function, and disease pathogenesis. For example, in the context of cancer, intratumor heterogeneity is a key indicator of disease progression, metastasis, and the development of drug resistance. High-throughput single cell analysis methods such as flow cytometry, mass cytometry, and single cell RNA sequencing (scRNA-seq) are ideal for identifying single cells in a comprehensive manner based on molecular information, and these methods have already begun to transform our understanding of complex tissues by enabling identification of previously unknown cell types and states.

However, a critical barrier to these efforts is the need to first process tissues into a suspension of single cells. Current methods involve mincing, digestion, disaggregation, and filtering that are labor intensive, time-consuming, inefficient, and highly variable. Thus, new approaches and technologies are critically needed to ensure reliability and wide-spread adoption of single cell analysis methods for tissues. This would be particularly important for translating single cell diagnostics to human specimens in clinical settings. Moreover, improved tissue dissociation would make it faster and easier to extract primary cells for ex vivo drug screening, engineered tissue constructs, and stem/progenitor cell therapies. Patient-derived organ-on-a-chip models, which seek to recapitulate complex native tissues for personalized drug testing, are a particularly exciting future direction that could be enabled by improved tissue dissociation.

scRNA-seq has recently emerged as a powerful and widely adaptable analysis technique that provides the full transcriptome of individual cells. This has enabled comprehensive cell reference maps, or atlases, to be generated for normal and diseased tissues, as well as identification of previously unknown cell subtypes or functional states. For example, an atlas recently generated for normal murine kidney uncovered a new collecting duct cell with a transitional phenotype and unexpected level of cellular plasticity. Moreover, an atlas of primary human breast epithelium linked distinct epithelial cell populations to known breast cancer subtypes, suggesting that these subtypes may develop from different cells of origin. For melanoma, scRNA-seq was used to identify three transcriptionally distinct states, one of which was drug sensitive, and further demonstrated that drug resistance could be delayed using computationally optimized therapy schedules. While scRNA-seq is clearly a powerful diagnostic modality, the mechanical process of breaking down the tissue into single cells can introduce confounding factors that may negatively influence data quality and reliability. One factor is the lack of standardization, which can lead to substantial variation across different research groups and tissue types. Another significant concern is that incomplete break down could bias results towards cell types that are easier to liberate. A recent study utilizing single nuclei RNA sequencing (snRNA-seq) with murine kidney samples found that endothelial cells and mesangial cells were underrepresented in scRNA-seq data. Finally, lengthy enzymatic digestion times have been shown to alter transcriptomic signatures and generate stress responses that interfere with cell classification. Addressing these concerns would help propel the exciting field of scRNA-seq into the future for tissue atlasing and disease diagnostics.

Microfluidic technologies have advanced the fields of biology and medicine by miniaturizing devices to the scale of cellular samples and enabling precise sample manipulation. Most of this work has focused on manipulating and analyzing single cells. Only a small number of studies have addressed tissue processing, and even fewer have focused on breaking down tissue into smaller constituents. For example, microfluidic devices have been developed that specifically focused on breaking down cellular aggregates into single cells. This dissociation device contained a network of branching channels that progressively decreased in size down to ˜100 μm, and contained repeated expansions and constrictions to break down aggregates using shear forces. Details regarding such devices may be found in Qiu, X. et al., Microfluidic device for mechanical dissociation of cancer cell aggregates into single cells, Lab Chip 15, 339-350 (2015) and Qiu, X. et al., Microfluidic channel optimization to improve hydrodynamic dissociation of cell aggregates and tissue, Nat. Sci. Reports 8, 2774 (2018).

A device was then developed for on-chip tissue digestion using the combination of shear forces and proteolytic enzymes. Finally, a filter device was developed containing nylon mesh membranes that removed large tissue fragments, while also dissociating smaller cell aggregates and clusters. See Qiu, X. et al., Microfluidic filter device with nylon mesh membranes efficiently dissociates cell aggregates and digested tissue into single cells, Lab Chip 18, 2776-2786 (2018). The microfluidic digestion, dissociation, and filter devices each enhanced single cell recovery when operated independently. To date, however, these technologies have not been combined to maximize performance and execute a complete tissue processing workflow on-chip. Moreover, there has been no validations of microfluidically-processed cell suspensions using scRNA-seq.

### SUMMARY

In one embodiment, a microfluidic platform or system is disclosed that includes three different tissue-processing technologies (digestion, disaggregation, and filtration) that enhances break-down and produces single cell suspensions that are immediately ready for downstream single cell analysis or other use. First, the system uses a digestion device that can be loaded with minced tissue and operated with minimal user interaction. Next, in a separate device that is fluidically coupled to the digestion device integrates or combines tissue dissociation and filter technologies into a single unit. The two-device platform was optimized using murine kidney to produce single cells more quickly and in higher numbers than traditional methods. Using the optimized protocol, different tissue types were evaluated using two single cell analysis methods. For murine kidney and breast tumor tissues, microfluidic processing can produce ˜2.5-fold more epithelial cells and leukocytes, and >5-fold more endothelial cells, without affecting viability. Using scRNA-seq, it was shown that device processed samples are highly enriched for endothelial cells, fibroblasts, and basal epithelium. It was also demonstrated that stress responses are not induced in any cell type, and can even be reduced if shorter processing times are employed. For murine liver and heart, significant single cell numbers are obtained after only 15 min, and even as short as 1 minute. Interestingly, it was found that substantially more hepatocytes and cardiomyocytes are obtained if sample is recovered at discrete intervals, most likely because these cell types are sensitive to shear forces. Importantly, the microfluidic platform can significantly shorten processing time or enhance single cell recovery for all tissue types studies, and in some cases accomplish both, without affecting viability. Furthermore, the entire tissue processing workflow is performed in an automated and reliable fashion. Thus, the microfluidic platform holds exciting potential to advance diverse applications that require the liberation of single cells from tissues.

In one embodiment, a microfluidic system for processing a tissue sample is disclosed that digests, dissociates, and optionally filters tissue. The system includes a microfluidic digestion device having an inlet and an outlet and a flow path defined between the inlet and the outlet, the flow path comprising a tissue chamber configured to hold the tissue sample and a plurality of upstream fluidic channels communicating with the tissue chamber on the inlet side of the flow path and a plurality of downstream fluidic channels communicating with the tissue chamber on the outlet side of the flow path. A first pump is configured to pump a buffer-containing fluid and/or an enzyme-containing fluid into the inlet of the microfluidic digestion device (while the tissue is present in the tissue chamber). The system further includes a microfluidic dissociation/filter device comprising an inlet, a first outlet, a second outlet, and a flow path defined between the inlet and the outlet, the flow path comprising a plurality of furcating dissociation channels having a plurality of expansion and constriction regions disposed along a length thereof, wherein one or more filters are disposed in the flow path downstream of the plurality of furcating dissociation channels. Either of the first and second outlets may be selectively closed to permit flow through the dissociation region of the device only or flow through the dissociation region of the device plus the filter region(s) of the device. A second pump is configured to pump a buffer-containing fluid into the inlet of the microfluidic dissociation/filter device (along with processed tissue solution from the microfluidic digestion device). The outlet of the microfluidic digestion device is fluidically coupled to the inlet of the microfluidic dissociation/filter device. Thus, fluid containing digested tissue passes from the microfluidic digestion device to the microfluidic dissociation/filter device.

In one embodiment, a method of using the microfluidic system includes the operations of: loading the tissue sample into the tissue chamber of the microfluidic digestion device; pumping the buffer-containing fluid and/or an enzyme-containing fluid into the inlet of the microfluidic digestion device with the first pump; transferring fluid containing processed tissue sample to the microfluidic dissociation/filter device; pumping the buffer-containing fluid into the inlet of the microfluidic dissociation/filter device along with the tissue processed with the microfluidic dissociation device; and collecting effluent from the second outlet of the microfluidic dissociation/filter device.

### BRIEF DESCRIPTION OF THE DRAWINGS

FIG. 1A schematically illustrates the microfluidic system for processing tissue. Processing of tissue involves digestion, dissociation/disaggregation, and filtration of tissue. The system includes a digestion device that first processes minced tissue. A first pump is used to pump buffer and/or enzyme solution through the digestion device while the minced tissue is contained in a tissue chamber in the digestion device. Fluidic channels direct hydrodynamic shear forces and proteolytic enzymes, while also retaining minced tissue pieces in the chamber. A dissociation/filter device is fluidically coupled to the output of the digestion device via valves (V). The dissociation/filter device includes a series of furcating (e.g., bifurcating) channels of smaller dimension along with expansion/contraction regions for imparting shear forces on the tissue fragments and cell aggregates. The dissociation/filter device further includes one or more filter media (e.g., two filter membranes) that are located in the flow path to filter out larger sized tissue fragments and cell aggregates. A separate pump is coupled to the dissociation/filter device via valves (V) and pumps buffer or other fluid into the dissociation/filter device along with the output of the digestion device. Single cells are output from the dissociation/filter device via an output.

FIG. 1B illustrates the dissociation channels formed in the digestion device. Note that these different stages of dissociation channels may be formed in different layers of a multi-layered device. Expansion and constriction regions are illustrated.

FIG. 1C illustrates a photographic of the experimental setup including the peristaltic pump, digestion device, dissociation/filter device, and connections via valving and tubing. In this photograph, the system recirculates fluid through the digestion device.

FIG. 1D illustrates schematically the configuration of the experimental setup of FIG. 1C. Here, the system is configured to recirculate fluid through the digestion device. A stopcock valve is used to divert flow back to the pump for recirculation.

FIG. 1E illustrates a photographic of the experimental setup including the peristaltic pump, digestion device, dissociation/filter device, and connections via valving and tubing. In this photograph, the system elutes sample during intervals or at the end of a run.

FIG. 1F illustrates schematically the configuration of the experimental setup of FIG. 1E. Here, the system is configured to pass fluid through the digestion device and the dissociation/filter device. This is used to elute cells. Fresh enzyme solution was used for intervals and buffer was used for the final elution.

FIG. 2A illustrates a schematic of the digestion device according to one embodiment. The design includes six (6) total layers, including two fluidic layers, 2 via layers, and the top and bottom end caps. Tissue is loaded through the luer port and into the tissue chamber.

FIG. 2B illustrates a schematic of the tissue chamber. Fluidic channels direct hydrodynamic shear forces and proteolytic enzymes, while also retaining minced tissue pieces in the chamber.

FIG. 2C illustrates photographs of the fabricated digestion device.

FIG. 2D illustrates a schematic of the integrated dissociation/filter device. Tissue fragments and cell aggregates from the digestion device will be further broken down by hydrodynamic shear forces generated in the furcating microchannels with the expansion/contraction regions and nylon mesh filters.

FIG. 2E illustrates a photograph of the fabricated dissociation/filter device.

FIG. 2F illustrates an exploded view of a digestion device according to another embodiment.

FIGS. 3A-3F: Device optimization using murine kidney. (FIG. 3A) Kidneys were harvested, minced, and processed using the minced digestion device at 10 or 20 m/min flow rate for 15 or 60 min, and total genomic DNA (gDNA) was quantified. The gDNA was extracted directly from the control, and thus this represents maximum recovery. Results at 20 m/min flow rate were superior, particularly at the shorter time point. (FIG. 3B) Pictures of tissue within the minced digestion device chamber before and after 15 or 60 min of processing at 10 (i) or 20 (ii) mL/min flow rate. Significant tissue remained at 10 mL/min, while tissue was larger absent at 20 mL/min. (FIG. 3C) Single EpCAM+ epithelial cells were quantified by flow cytometry after samples were processed with the minced digestion device for 15, 30, or 60 min. The recovery of sample at different time intervals was also evaluated, with more collagenase added to continue processing of remaining tissue. (FIG. 3D) Epithelial cell viability was ˜80% for all control and device conditions. (FIG. 3E) Samples were processed with the integrated dissociation/filter device following 15 min of digestion device treatment. A single pass through the integrated device produced optimal results. (FIG. 3F) Epithelial cell viability was at ˜85-90% for all conditions. Error bars represent standard errors from at least three independent experiments. * indicates p<0.05 and ** indicates p<0.01 relative to the control at the same digestion time. # indicates p<0.05 relative to the static condition at the same digestion time. Scale bar represents 5 mm.

FIGS. 4A-4C: Microfluidic platform results for murine kidney. Kidneys were harvested, minced, processed with the digestion device for different 15 or 60 min, passed through the integrated dissociation/filtration device one time, and resulting cell suspensions were analyzed using flow cytometry. Interval recovery was evaluated at 1-, 15-, and 60-min time points from the same tissue sample. Controls were minced, digested for either 15 or 60 min, pipetted/vortexed, and passed through a cell strainer. (FIG. 4A) Single EpCAM+ epithelial cells increased by 2.5-fold with microfluidic processing. Interval results were comparable to static, and the 1 min interval produced comparable cell numbers to the 15 min control. Trends were similar for (FIG. 4B) endothelial cells and (FIG. 4C) leukocytes. Microfluidic processing was particularly effective for endothelial cells, yielding >5-fold more cells than the control at 60 min. Endothelial cells were enriched for all device conditions except the 1 min interval relative to controls. Error bars represent standard errors from at least three independent experiments. * indicates p<0.05 and ** indicates p<0.01 relative to the control at the same digestion time.

FIGS. 5A-5C: scRNA-seq of murine kidney. Cell suspensions obtained from the microfluidic platform at 15- and 60-min intervals, as well as the 60 min control, were sorted by FACS to remove dead cells and debris, loaded onto a 10× Chromium chip, and sequenced at >50,000 reads/cell. (FIG. 5A) UMAP displaying seven cell clusters that correspond to two different proximal tubule sub-types, endothelial cells, macrophages, B lymphocytes, T lymphocytes. The seventh cluster contained a mixed population corresponding to distal convoluted tubules (DCT), loop of Henle (LOH), collecting duct (CD), and mesangial cells (MC). (FIG. 5B) Population distributions for each cell cluster and processing condition. Proximal tubules were predominantly eluted from the microfluidic platform in the 15 min interval, while endothelial cells and macrophages were enriched in the 60 min interval. (FIG. 5C) Stress response scores were generally lower for the 15 min device interval.

FIGS. 6A-6C: Microfluidic platform results for murine breast tumor. Breast tumors from MMTV-PyMT mice were resected, minced, processed with the microfluidic platform, and analyzed by flow cytometry. (FIG. 6A) EpCAM+ epithelial cells were ˜2-fold higher at both time points. (FIG. 6B) Endothelial cells were enhanced even more by the microfluidic platform, with 5- and 10-fold more cells were recovered after 15 min and 60 min, respectively. (FIG. 6C) Leukocytes increased by 3- and 5-fold after 15 and 60 min, respectively. The interval format produced similar total cell numbers relative to the corresponding static time point, except for endothelial cells, which were slightly higher. Device processing enriched both endothelial cells and leukocytes at all but the 1 min time point. Error bars represent standard errors from at least three independent experiments. * indicates p<0.05 and ** indicates p<0.01 relative to the control at the same digestion time. # indicates p<0.05 and ## indicates p<0.01 relative to the static condition at the same digestion time.

FIGS. 7A-7C: scRNA-seq of murine mammary tumor. Cell suspensions obtained from the microfluidic platform at 15- and 60-min intervals, as well as the 60 min control, were processed and analyzed using similar methods to kidney. (FIG. 7A) UMAP displaying six cell clusters that correspond to epithelial cells, macrophages, endothelial cells, T lymphocytes, fibroblasts, and granulocytes. (FIG. 7B) Population distributions for each cell cluster and processing condition. Epithelial cells were predominantly eluted from the microfluidic platform in the 15 min interval, while endothelial cells and fibroblasts were enriched in the 60 min interval. Fibroblasts were enriched in both platform conditions, while granulocytes were depleted. (FIG. 7C) Stress response scores were generally similar across conditions and cell types.

FIGS. 8A-8E: Microfluidic platform results for murine liver. (FIGS. 8A, 8B) Liver was harvested, minced, and evaluated with the minced digestion device alone and in combination with the integrated dissociation/filter device. Hepatocytes were identified and quantified by flow cytometry. (FIG. 8A) The digestion device increased hepatocyte recovery by ˜4-fold at 15 min, but continued digestion and passing through the integrated dissociation/filter device one-time decreased hepatocyte yield, likely due to the large size and fragile nature of hepatocytes. (FIG. 8B) Hepatocyte viability was ˜75-80% for all conditions, except the 60 min integrated condition. (C-F) Results using shorter digestion times and a single pass with a dissociation/filtration device containing only the 50 μm filter. (FIG. 8C) After only 5 min of microfluidic processing, 4-fold more cells were obtained than the 15 min control and only slightly less than the 60 min control. Interval recovery enhanced hepatocyte yield by ˜2.5-fold relative to the 60 min control and 15 min static conditions. The 1-minute interval contributed substantially, producing ˜70% as many hepatocytes as the 60 min control. Similar results were observed for (FIG. 8D) endothelial cells and (FIG. 8E) leukocytes, although the benefit of intervals was less pronounced. Microfluidic processing generally enriched for leukocytes, although there was a shift to hepatocytes for the later intervals. Error bars represent standard errors from at least three independent experiments. * indicates p<0.05 and ** indicates p<0.01 relative to the 60 min control at the same digestion time. # indicates p<0.05 and ## indicates p<0.01 relative to the static condition at the same digestion time.

FIGS. 9A-9C: Microfluidic platform results for murine heart. Hearts were resected, minced, processed with the microfluidic platform (both 50 and 15 μm membranes), and analyzed by flow cytometry. Shorter digestion device time points were employed due to the sensitivity of cardiomyocytes. (FIG. 9A) Microfluidic processing produced ˜12,000 cardiomyocytes per mg after 15 min, which was ˜2-fold higher than the 60 min control. Interval recovery produced optimal results again, increasing by ˜50% and ˜3-fold relative to the 15 min static and 60 min control conditions. (FIG. 9B) Endothelial cell and (FIG. 9C) leukocyte yields were significantly lower than the 60 min control under both static and interval formats. Interval recovery did improve, but remained ˜2-fold lower than the 60 min controls. Microfluidic processing generally enriched for cardiomyocytes. Error bars represent standard errors from at least three independent experiments. * indicates p<0.05 and ** indicates p<0.01 relative to the 60 min control at the same digestion time. # indicates p<0.05 and ## indicates p<0.01 relative to the static condition at the same digestion time.

FIGS. 10A-10F: Recirculation studies with MCF-7 cell line. MCF-7 breast cancer cells were continuously pumped through the (FIGS. 10A, 10D) peristaltic pump, (FIGS. 10B, 10E) minced digestion device, or (FIGS. 10C, 10F) dissociation/filter device at different flow rates and for different time periods. (FIGS. 10A-10C) Cell counts were obtained and normalized to the control. Cell numbers decreased modestly for (FIG. 10A) pump alone and (FIG. 10B) digestion device under all conditions. (FIG. 10C) The dissociation device increased cell recovery for the longer time points at 10 mL/min and all time points at 20 mL/min. (FIGS. 10D-10F) Cell viability remained high for (FIG. 10D) pump only, (FIG. 10E) digestion device, and (FIG. 10F) dissociation device at 5 mL/min. However, higher flow rates decreased viability for the dissociation device, in a manner that correlated inversely with increases in single cell yield. Error bars represent standard error from at least three independent experiments. * indicates p<0.05 and ** indicates p<0.01 relative to the control.

FIGS. 11A-11D: Leukocyte results from optimization studies using murine kidney. (FIGS. 11A, 11B) Minced digestion device optimization under static and interval formats, compared to control that was digested for 60 min. (FIG. 11A) Leukocyte yield increased with digestion device processing time to ˜1000/mg, exceeding the control by ˜30%. Interval recovery did not affect results. (FIG. 11B) Viability increased from ˜65% for control to >70% for all device conditions. (FIGS. 11C, 11D) Integrated dissociation/filter device optimization using sample that was processed for 15 min in the digestion device, compared to control digested for 15 min. (FIG. 11C) Leukocyte recovery remained the same after a single pass and decreased modestly with recirculation. (FIG. 11D) Leukocyte viability was ˜85-90% for all conditions. Error bars represent standard error from at least three independent experiments. * indicates p<0.05 and ** indicates p<0.01 relative to the control. # indicates p<0.05 relative to the static condition at the same digestion time.

FIG. 12: Red blood cell results for murine kidney. Most RBCs were eluted at early timepoints for device processing. Due to the high recovery after only 1 min, this time point was added to interval studies for all tissues. Error bars represent standard error from at least three independent experiments. * indicates p<0.05 relative to the control at the same digestion time.

FIGS. 13A-13C: Cell viability from final microfluidic platform studies using murine kidney. (FIG. 13A) Epithelial cell viability was ˜95% for all conditions. (FIG. 13B) Endothelial cell and (FIG. 13C) leukocyte viabilities ranged from ˜60% to 90%, with the 60 min control at ˜70% in both cases. Device platform processing resulted in higher viabilities for endothelial cells at all conditions except the 1 min interval, and leukocytes were elevated at the 15 min time points (static and interval). Error bars represent standard error from at least three independent experiments. * indicates p<0.05 and ** indicates p<0.01 relative to the control at the same digestion time. # indicates p<0.05 relative to the static condition at the same digestion time.

FIGS. 14A-14B: Gene scoring of kidney cell types. Cell scoring results that were used to compare marker gene signatures for each of the (FIG. 14A) seven main clusters and (FIG. 14B) sub-clusters.

FIG. 15A: UMAP representation showing the 4 sub-cluster within the DCT, LOH, CD, & MC cluster.

FIG. 15B: Distributions obtained for each sub-cluster of FIG. 15A, relative to the full population.

FIGS. 16A-16D: Expression of EpCAM, CD45, and CD31 in kidney clusters. (FIGS. 16A-B) EpCAM was highly expressed within the (FIG. 16A) DCT, LOH, CD, & MC cluster and (FIG. 16B) each individual sub-cluster. (FIG. 16C) CD45 was highly expressed in the macrophage, B lymphocyte, and T lymphocyte clusters. (FIG. 16D) CD31 was highly expressed in the endothelial cluster.

FIGS. 17A-17D: Device optimization studies using murine breast tumor. (FIGS. 17A, 17B) Minced digestion device operated for different time points. (FIG. 17A) Epithelial cell yield increased by ˜2- to 2.5-fold using the digestion device. (FIG. 17B) Viability was ˜80% for the 15 min control and decreased slightly with time, while all device conditions were >85%. (FIGS. 17C, 17D) Integrated dissociation/filter device optimization using sample that was processed for 15 min in the digestion device. (FIG. 17C) Epithelial recovery increased by 30% after a single pass, while recirculation produced similar or lower numbers. (FIG. 17D) Viability decreased slightly after dissociation/filter treatment, but changes were not significant. Error bars represent standard error from at least three independent experiments. * indicates p<0.05 relative to the control at the same digestion time.

FIGS. 18A-18C: Cell viability from final microfluidic platform studies using murine breast tumor. (FIG. 18A) Epithelial cell viability was ˜70-80% for all conditions. (FIG. 18B) Endothelial cell viability was generally low at ˜50-60%. However, the 1 min device interval was higher at 75%, while the 60 min control and 15 min device interval were lower at 50% and 40%, respectively. (FIG. 18C) Leukocyte viability remained ˜80% for all but the 60 min control, which was ˜60%. Error bars represent standard error from at least three independent experiments. * indicates p<0.05 and ** indicates p<0.01 relative to the control at the same digestion time. # indicates p<0.05 relative to the static condition at the same digestion time.

FIGS. 19A-19C: Sub-clustering epithelial cells for murine breast tumor. (FIG. 19A) The epithelial cluster contained 3 distinct sub-clusters that corresponded to luminal, basal, and proliferating luminal. (FIG. 19B) Population distributions in each sub-cluster. Luminal cells were enriched in the 15 min interval, while basal cells were enriched at 60 min. (FIG. 19C) The sub-clusters were identified primarily based on expression of Krt14 (basal), Krt18 (luminal), and Mki67 (proliferating) genes.

FIGS. 20A-20D: Expression of EpCAM, CD45, and CD31 in breast tumor clusters. (FIGS. 20A-B) EpCAM was highly expressed within the (FIG. 20A) epithelial cluster and (FIG. 20B) each sub-cluster. (FIG. 20C) CD45 was highly expressed in the macrophage, T lymphocyte, and granulocyte clusters. (FIG. 20D) CD31 was highly expressed in the endothelial cluster.

FIGS. 21A-21B: Device optimization studies using murine liver. Liver was processed with the minced digestion for 15 min and passed through the modified dissociation/filter device (50 μm filter only). (FIG. 21A) Hepatocytes increased by 30% relative to the digestion device alone and by nearly 3-fold relative to 15 min control. (FIG. 21B) Hepatocyte viability was >85% for all conditions. Error bars represent standard error from at least three independent experiments.

FIGS. 22A-22C: Cell viability from final microfluidic platform studies using murine liver. (FIG. 22A) Hepatocyte viability remained ˜90% for all conditions except the 60 min interval, which decreased to ˜85%. (FIG. 22B) Endothelial cell and (FIG. 22C) leukocyte viabilities were generally between ˜70% and 85%, and increased with device processing at the early time points. Error bars represent standard error from at least three independent experiments. * indicates p<0.05 and ** indicates p<0.01 relative to the 60 min control. # indicates p<0.05 and ## indicates p<0.01 relative to the static condition at the same digestion time.

FIGS. 23A-23B: Device optimization studies using murine heart. Heart was processed with the minced digestion device for 15 min and passed through the integrated dissociation/filter with the original (50 and 15 μm filters) or modified (50 μm filter only) format. (FIG. 23A) Cardiomyocyte yield and (FIG. 23B) viability were similar for all conditions. Error bars represent standard error from at least three independent experiments.

FIGS. 24A-24C: Cell viability from final microfluidic platform studies using murine heart. (FIG. 24A) Cardiomyocyte viability for device processed samples matched or exceeded controls. (FIG. 24B) Endothelial cell and (FIG. 24C) leukocyte viability was generally >80% for device and control conditions. Error bars represent standard error from at least three independent experiments. * indicates p<0.05 and ** indicates p<0.01 relative to the 60 min control.

FIG. 25: Flow cytometry gating schemes. Cell suspensions were stained fluorescent probes (listed in Table 1) and signals were assessed by flow cytometry. Data was then analyzed using a sequential gating scheme. Gate 1 used FSC-A vs. SSC-A to exclude debris near the origin. Gate 2 used FSC-A vs. FSC-H to select single cells. Gate 3 used CD45-BV510 vs. TER119-AF647 to distinguish leukocytes (CD45+TER119−) and red blood cells (CD45−TER119+). Gate 4 was applied to the CD45−TER119− subset, and used PE to identify epithelial cells via EpCAM (kidney and tumor), hepatocytes via ASGPR1 (liver), or cardiomyocytes via Troponin T (heart). Gate 5 was applied to the EpCAM/ASGPR1/Troponin T negative cell subset and used CD31-AF488 to identify endothelial cells. Finally, gate 6 used 7-AAD (kidney, tumor, liver) or Zombie Violet (heart) to distinguish live and dead cells.

FIGS. 26A-26B illustrate Podocyte markers. Gene expression of (FIG. 26A) Nphs1 and (FIG. 26B) Nphs2, with positive expression showing in only a small number of cells that were predominantly in the LOH, DCT, CD, & MC cluster.

FIGS. 27A-27L illustrate the expression of select stress response genes for each kidney cell cluster. Average gene expression for common stress response genes including (FIG. 27A) Nr4a1, (FIG. 27B) Gadd45b, (FIG. 27C) Atf3, (FIG. 27D) Egr1, (FIG. 27E) Jun, (FIG. 27F) Junb, (FIG. 27G) Jund, (FIG. 27H) Fos, (FIG. 27I) Fosb, (FIG. 27J) Hsp90aa1, (FIG. 27K) Hspa8, and (FIG. 27L) Hspd1.

FIGS. 28A-28L illustrate the expression of select stress response genes for each breast tumor cell cluster. Average gene expression for common stress response genes including (FIG. 28A) Nr4a1, (FIG. 28B) Gadd45b, (FIG. 28C) Atf3, (FIG. 28D) Egr1, (FIG. 28E) Jun, (FIG. 28F) Junb, (FIG. 28G) Jund, (FIG. 28H) Fos, (FIG. 28I) Fosb, (FIG. 28J) Hsp90aa1, (FIG. 28K) Hspa8, and (FIG. 28L) Hspd1.

### DETAILED DESCRIPTION OF ILLUSTRATED EMBODIMENTS

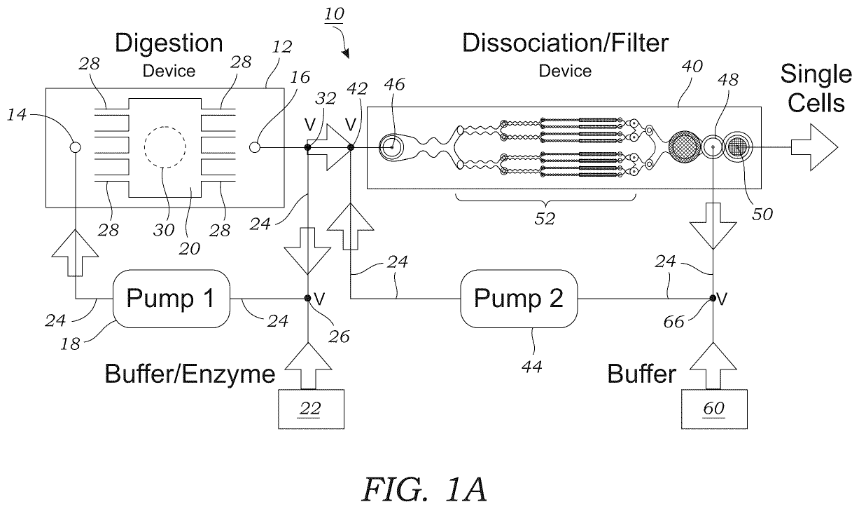

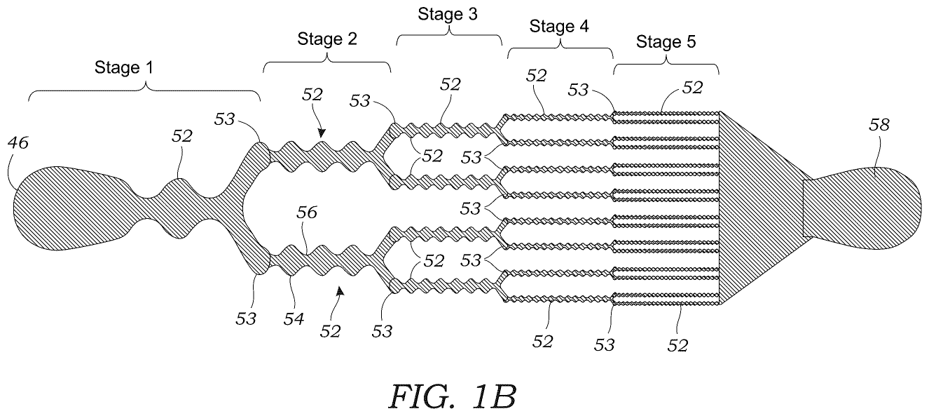

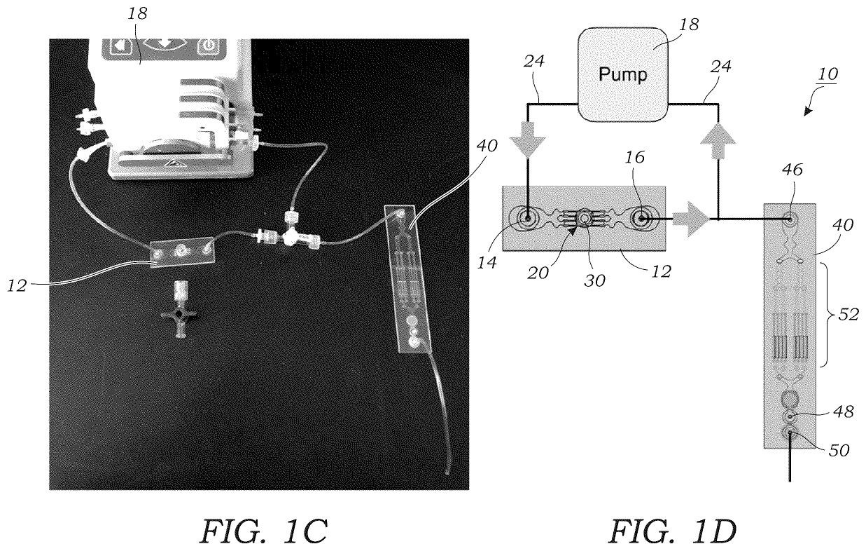

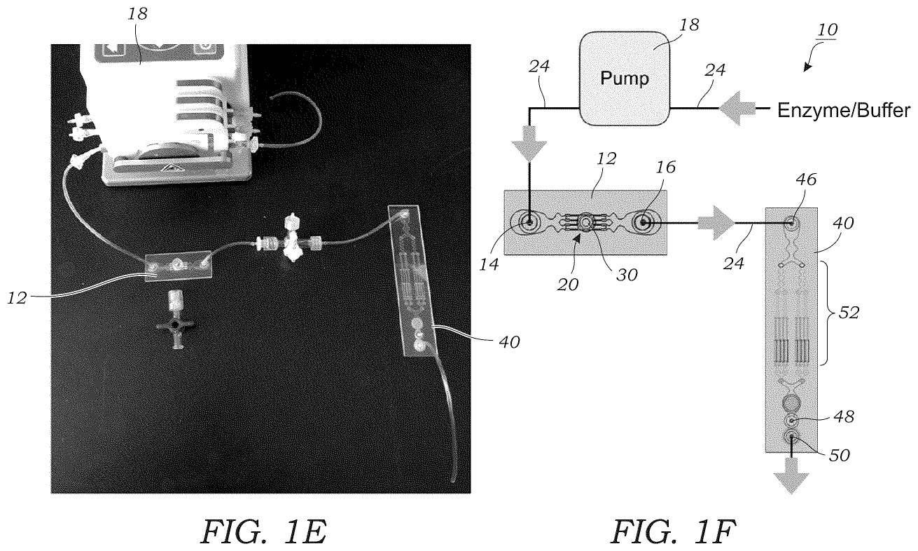

FIGS. 1A, 1D, and 1F schematically illustrates a microfluidic system 10 for processing tissue. The microfluidic system 10 includes a digestion device 12 that first processes minced tissue. The digestion device 12 includes an inlet 14 and an outlet 16. Barbs may be located at the inlet 14 and outlet 16 so that tubing (e.g., tubing or conduit 24 as described herein) may be readily secured thereto to that fluid can be flowed into the digestion device 12 as well as removed from the digestion device 12. With reference to FIG. 1A, a first pump 18 (e.g., peristaltic pump) is used to pump buffer and/or enzyme solution through the digestion device 12 while the minced tissue is contained in a tissue chamber 20 in the digestion device. The first pump 18 is fluidically coupled to a source of fluid 22 that contains the buffer and/or enzyme solution via tubing or conduit 24. As explained herein, in some embodiments such as those illustrated in FIGS. 1C-1F only a single pump 18 is used. In other embodiments such as that illustrated in FIG. 1A, uses a first pump 18 and a second pump 44, the operation of which is explained further herein. A first valve 26 is interposed in the tubing or conduit 24, which as explained herein may be used to toggle fluid from between the source of fluid 22 and return from the digestion device 12.

The tissue chamber 20 may be square or rectangular shaped. An exemplary size for the tissue chamber 20 may be chamber that has a 5 mm length and 8 mm width with a height of 1.5 mm. In other embodiments, the tissue chamber 20 may be larger to accommodate larger tissue samples. For example, the tissue chamber 30 may be rectangular-shaped and dimensioned to accommodate a sliver or larger piece of tissue. The tissue chamber 30 may have a length of several centimeters (e.g., around 2-3 cm) and even up to about 10 cm. The first pump 18 may be connected to the digestion device 12 via tubing or conduit 24 as illustrated in FIG. TA. In response to fluid flow into the digestion device 12 via the first pump 18, fluidic channels 28 direct hydrodynamic shear forces and proteolytic enzymes (if contained in fluid), while also retaining minced tissue pieces in the tissue chamber 20. In one embodiment, the number of upstream fluidic channels 28 are equal to the number of downstream fluidic channels 28 (e.g., four such fluidic channels 28 are illustrated in FIG. TA). In this embodiment, the upstream fluidic channels 28 are symmetrical with the downstream fluidic channels 28. Other numbers of fluidic channels 28 may be used. The width of the upstream/downstream fluidic channels 28 may be the same in some embodiments. An exemplary width of the fluidic channels 28 is about 250 μm, although as explained herein other dimensions of the fluidic channels 28 may be used. The length of the fluidic channels 28 may be several millimeters (e.g., 4 mm). The fluidic channels 28 are separated from one another by about 1 mm to ensure reliable fabrication and integrity. The fluidic channels 28 enlarge in the region where they join the underlying via layer (i.e., flare), which was also intended to prevent clogging.

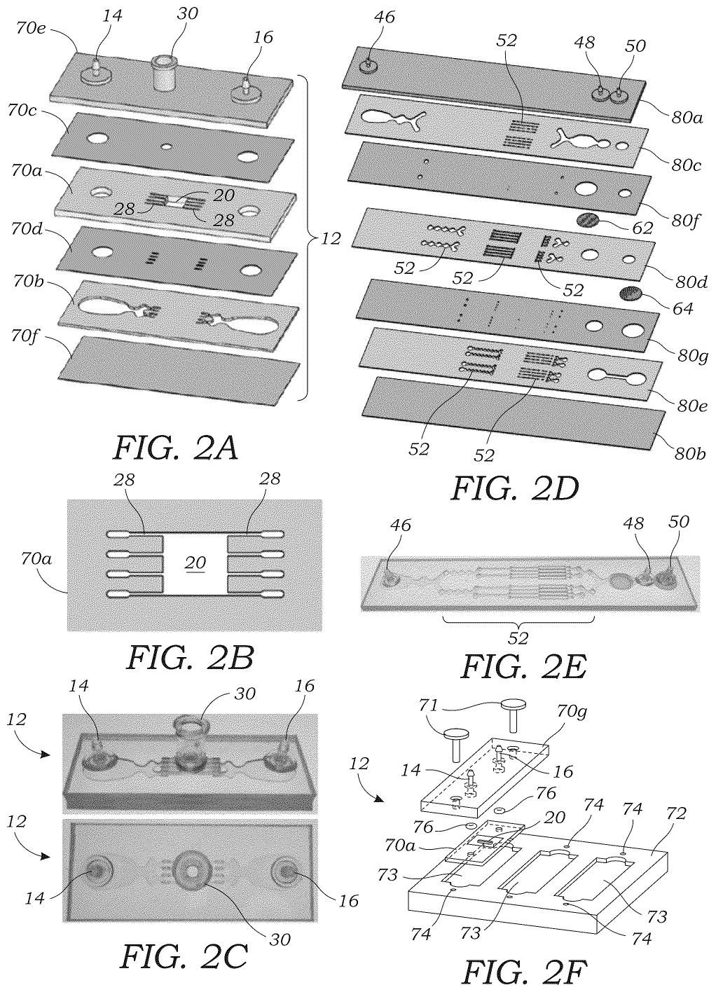

The number of fluidic channels 28 may vary depending on the size of the tissue chamber 20. For example, FIG. 2B illustrates four (4) upstream fluidic channels 28 and four (4) downstream fluidic channels 28. In other embodiments where the tissue chamber 20 has a larger volume or size, the number of fluidic channels 28 may be larger. For example, in other embodiments where a strip or larger piece of tissue is inserted into the tissue chamber 20, there may be between 10-20 fluidic channels 28 on the upstream/downstream sides of the tissue chamber 20. Likewise, the width of the fluidic channels 28 may vary. For some embodiments (like where larger tissue pieces are processed), the width of the fluidic channels 28 may be larger, for example, having a width in the range between about 500 μm and about 750 μm.

The tissue sample (e.g., minced tissue) is loaded into the digestion device 12 via a port 30 (e.g., luer port) as seen in FIGS. 1A, 2A, 2C or by opening the top layer 70g (FIG. 2F) as described below. The port 30 can be closed with a plug or stopcock after loading. As seen in FIG. 1A, the first pump 18 pumps buffer and/or enzyme(s) through the digestion device 12. The first valve 26 is used to modulate flow of buffer and/or enzyme(s) into the digestion device 12. The digestion device 12 may also run in a recirculation mode so that the output of the digestion device 12 is pumped back through the digestion device 12 (i.e., the output from outlet 16 is pumped back into the inlet 14 via the first pump 18). In the recirculation mode, the first valve 26 and a second valve 32 is actuated to allow flow of fluid (and contents contained therein) to recirculate through the digestion device 12. Of course, the digestion device 12 may also be configured in a mode to not recirculate flow back into the digestion device 12. In this configuration, the output of the outlet 16 proceeds to the dissociation/filter device 40 as described below. In this later mode, the second valve 32 is actuated to direct flow to the dissociation/filter device 40.

Still referring to FIG. 1A, the dissociation/filter device 40 is fluidically coupled to the output of the digestion device 12 via tubing or conduit 24 along with a third valve 42 interposed between the digestion device 12 and the dissociation/filter device 40. The third valve 42 is actuated to either allow flow to enter the dissociation/filter device 40 from the outlet 16 of the digestion device 12 or from a second pump 44. The dissociation/filter device 40 includes an inlet 46, a first outlet 48, and a second outlet 50. The inlet 46, first outlet 48, and second outlet 50 may have barbed ends to facilitate attaching tubing or conduit 24 thereto. The first outlet 48 is used to pass processed tissue fragments and cell aggregates that have been subject to dissociation forces from the series of dissociation channels 52 formed in the dissociation/filter device 40 but not otherwise filtered. The dissociation channels 52 are formed by a series of furcating (e.g., bifurcating) channels of smaller dimension (e.g., width) in the direction of fluid flow along with expansion/contraction regions formed along their length for imparting shear forces on the tissue fragments and cell aggregates. For example, as seen in FIG. 1B, the dissociation channels 52 may include a single channel 52 that bifurcates into two (2) channels 52 at bifurcation 53, which then bifurcates into the four (4) channels 52 at bifurcations 53, which then bifurcates into eight (8) channels 52 at bifurcations 53, which then bifurcates into sixteen (16) channels 52 at bifurcations 53. In this embodiment, there are thus five (5) stages. The bifurcated dissociation channels 52 have a reduced width (½) as compared to the upstream dissociation channel 52. Along the length of the dissociation channels 52 are expansion regions 54 and constriction regions 56 that are configured to impart shear stresses on the tissue fragments and cell aggregates passing therethrough. The expansion and constriction regions 54, 56 are preferably continuous along the length of the dissociation channels 52. The expansion and constrictions regions 54, 56 in the dissociation channels 52 are alternating regions where the width of the dissociation channel 52 increases and decreases. The expansion and constrictions regions 54, 56 generate fluidic jets of varying size scales and magnitudes to help break down tissue fragments and cell aggregates using hydrodynamic shear forces. The design of the expansion and constrictions regions enables gradual disaggregation, thereby maximizing cell yield without causing extensive cell damage.

In one preferred embodiment, the width at the expansion regions 54 within a particular stage is 3× the width of the constriction region 56. Thus, in one embodiment, the first stage (single dissociation channel 52) has a constriction region 56 width that is 2 mm and width of the expansion region(s) 54 is 6 mm. In the second stage, the constriction region 56 width is 1 mm while the width of the expansion regions 54 is 3 mm. In the third stage, the constriction region 56 width is 0.5 mm while the width of the expansion regions 54 is 1.5 mm. In the fourth stage, the constriction region 56 width is 0.25 mm while the width of the expansion regions 54 is 0.75 mm. In the fifth stage, the constriction region 56 width is ˜0.125 mm while the width of the expansion regions 54 is ˜0.375 mm. After the last stage of dissociation channels 52, the channels collect fluid to a common collection region 58. As discussed below, the fluid containing the processed tissue may then be directed either out of the dissociation/filtration device 40 (without filtration) or through filter media for filtration.

Still referring to FIG. 1A, the dissociation/filter device 40 has two flow paths for fluid that contains the processed tissue fragments and cell aggregates. In a first flow path, the processed tissue fragments and cell aggregates that pass through the dissociation channels 52 passes through the first outlet 48. In this flow path, there is no filter media interposed in the flow path. For example, the processed tissue fragments and cell aggregates leave the first outlet 48 and then may be recirculated into the dissociation/filter device 40 using the first pump 18 and/or the second pump 44. This second pump 44 is used to pump buffer fluid from a buffer source 60 through the dissociation/filter device 40 (e.g., to flush the dissociation/filter device 40) and/or recirculate processed tissue fragments and cell aggregates back to the inlet 46.

In a second flow path, the processed tissue fragments and cell aggregates are then directed through two different filters 62, 64 (e.g., nylon mesh filters). The flow along the second flow path may be accomplished by plugging or capping the flow from the first outlet 48 which then forces the fluid (and contents) along the second flow path in response to pumping by first pump 18 and/or second pump 44. The first filter 62 in the flow path may have a larger pore size (e.g., ˜50-100 μm) than the second filter 64 (e.g., ˜15-50 μm) in the flow path to allow for first filtering of larger sized tissue fragments and cell aggregates followed by a smaller filter mesh with smaller pore size. Typically, the pores range in size from about 5 μm to about 1,000 μm and more preferably within the range from about 10 μm to about 1,000 μm or from about 5 μm to about 100 μm. In one embodiment, the first filter membrane 62 has pores having diameters of d₁ and the second filter membrane 64 has pores having diameters of d₂, wherein d₁>d₂. A second pump 44 is coupled to the dissociation/filter device 44 via conduits or tubing 24. A fourth valve 66 is provided to allow for the recirculation of flow from the dissociation/filter device 40 and also for adding buffer or other fluid into the dissociation/filter device 40. The second outlet 50 carries fluid that has passed through the filters 62, 64. This fluid typically contains single cells that are output from the dissociation/filter device 40.

FIG. 2A illustrates a schematic of the digestion device 12 according to one embodiment. The design includes six (6) total layers, including two fluidic layers 70a, 70b, two intermediate via layers 70c, 70d, and the top and bottom end caps 70e, 70f. Tissue is loaded through the luer port 30 and into the tissue chamber 20. FIG. 2C illustrates photographs of assembled digestion device 12. FIG. 2B illustrates a schematic of the tissue chamber 20 located in fluidic layer 70a. Fluidic channels 28 direct fluid which imparts hydrodynamic shear forces and carries proteolytic enzymes, while also retaining minced tissue pieces in the tissue chamber 20. FIG. 2D illustrates an exploded view of the layers used in the integrated dissociation/filter device 40. Tissue fragments and cell aggregates from the digestion device 12 are further broken down by hydrodynamic shear forces generated in the furcating dissociation channels 52 with the expansion/constriction regions 54,56 and nylon mesh filters 62, 64.

FIG. 2F illustrates another embodiment of the digestion device 12. In this embodiment, rather than load tissue using a port 30 as illustrated in FIGS. 2A-2C, the tissue chamber 20 is open and covered using a removable top layer 70g as seen in FIG. 2F. The fluidic channels 28 and the tissue chamber 20 are formed in layer 70a and a top layer 70g is used to cover and seal the tissue chamber 20 after loading of the tissue sample. A pair of fasteners 71 are used to secure and seal the top layer 70g against layer 70a. A thin plastic layer with adhesive on the backside may be used to seal the tissue chamber 20 prior to securing the top layer 70g against layer 70a. As seen in FIG. 2E, a base 72 is provided that includes recesses 73 dimensioned to accommodate one or more digestion devices 12. The fasteners 71, which may include threaded screws or the like, engage with apertures 74 in the base 72. In this regard, the digestion device 12 can be quickly loaded with tissue and then assembled for use. As seen in FIG. 2F, a pair of o-rings 76 are located in recesses in the top layer 70g where the inlet 14 and outlet 16 are located.

The dissociation/filter device 40 may also formed from multiple layers 80a-80g (e.g., seven layers). As seen in FIG. 2D, this includes a top layer 80a, bottom layer 80b, and intermediate fluidic channel layers 80c, 80d, 90e that contain the dissociation channels 52, along with flow paths for the optional filters 62, 64 as described herein. Via layers 80f, 80g are provided that includes holes or apertures for vertical flow and also flow paths that contain a first filter 62 and second filter 64. The digestion device 12 and the dissociation/filter device 40 can be fabricated using a commercial laminate process, with channel and via features laser micro-machined into hard plastic (Polymethyl methacrylate (PMMA) or Polyethylene terephthalate (PET)). All layers and other components can then be aligned and bonded using pressure sensitive adhesive. Photographs of the fabricated devices are shown in FIG. 2C (digestion device 12) and FIG. 2E (dissociation/filter device 40).

To use the system 10, a sample of tissue is placed in the tissue chamber 20. The tissue that is processed is preferably minced prior to placement in the tissue chamber 20 (e.g., scalpel mincing tissue into pieces with sizes of ˜1 mm³). The sample of tissue may include any type of mammalian tissue including, for example, kidney, liver, heart, mammary tissue. The tissue may be healthy or diseased. The digestion device 12 is then primed with buffer and enzyme with the first pump 18. The first pump 18 is preferably a peristaltic pump. The port 30 is then sealed with a stopcock or the like and fluid is then recirculated through the digestion device 12 with the first pump 18. The flow rate through the digestion device 12 may vary but is generally within the range of about 10 to about 100 mL/min. The recirculation may take place for several minutes to up to an hour or more. In some embodiments, the recirculation flow is maintained over this entire time period (i.e., static flow operation). In other embodiments, the digestion device 12 is run using an interval operation where the tissue is processed for short time periods, eluting the cell suspension, replacing the enzyme (e.g., collagenase) in the digestion device 12 and then continuing recirculation.

While the digestion device 12 disclosed herein uses a luer port 30 other ports may be used. In addition, in still other embodiments, the top layer 70a of the digestion device 12 may include a lid or cap that can be secured to the remainder of the digestion device 12 to load tissue inside the tissue chamber 20. The lid or cap may be secured using one or more fasteners or the like. Note that the device components of the system 10 (e.g., microfluidic digestion device 12 and dissociation/filter device 40) are preferably kept incubated in an incubator or temperature-controlled environment at about 37° C. to maintain optical enzymatic activity.

Once the sample has been processed with the digestion device 12, the now processed sample then moves to the dissociation/filter device 40. Fluid exits the outlet 16 and passes through tubing or conduit 24 and enters the inlet 46 of the dissociation/filter device 40. If recirculation is intended, tubing or conduit 24 connects the first outlet 48 (i.e., cross-flow outlet) to the second pump 44, while the second outlet 50 is closed off with a stopcock. Fluid is then pumped or recirculated through the dissociation channels 52 using second pump 44. This second pump 44 may include a syringe pump or a peristaltic pump. The flow rate through the dissociation/filter device 40 may vary but is generally within the range of about 5 to about 50 mL/min. For final collection of the sample, or if only a single pass through the dissociation component (i.e., dissociation channels 52) is utilized, the cross-flow outlet 48 is closed off with a stopcock valve (or cap/plug), and sample is pumped through and collected from the second outlet 50 (i.e., effluent outlet). This fluid or effluent contains single cells. The dissociation/filter device 40 may be washed with buffer to flush out and collect any remaining cells. Thus, for the dissociation/filter device 40, a single pass may be made through the dissociation channels 52 and the filters 62, 64 and out the second outlet 50. Alternatively, the sample from the digestion device 12 may recirculate through the dissociation channels 52 for a plurality of cycles followed by a pass through the filters 62, 64 and out the second outlet 50.

The microfluidic digestion device 12 and the dissociation/filter device 40 may be fluidically connected via tubing or conduit 24. Likewise, tubing or conduit 24 connect the pumps 18, 44 to the microfluidic digestion device 12 and the dissociation/filter device 40. The valves 26, 32, 42, 66 are interposed in the conduit or tubing 24 as illustrated, for example, in FIG. 1A. These valves 26, 32, 42, 66 and the pumps 18, 44 may be computer controlled using a control unit or computing device. For example, the control unit or computing device may control the flow rates of the pumps 18, 44 as well as the timing and actuation of the valves 26, 32, 42, 66.

### EXPERIMENTAL

Device Design and Fabrication

Minced tissue is loaded through a port at the top of the device 12, which can then be sealed using a cap or stopcock. Scalpel mincing of tissue into ˜1 mm³ pieces is ubiquitous, and therefore this format will be compatible with a wide array of tissue types and dissociation protocols. The full design layout of the new minced tissue digestion device is shown in FIG. 2A, including the loading port 30, a tissue chamber 20 that retains the tissue in place, and fluidic channels 28 that administer fluid shear forces and deliver proteolytic enzymes. These features were arranged across six layers 70a-70f of hard plastic, including two fluidic channel layers 70a, 70b, two “via” layers 70c, 70d, a top end cap 70e with hose barbs and loading port 30, and a bottom end cap 70f. The tissue chamber 20 is in the uppermost fluidic layer 70a, directly beneath the loading port 30 and a 2.5 mm diameter via, and a detailed schematic is shown in FIG. 2B. A square geometry was employed, with 5 mm length and width, to allow tissue to be evenly distributed during loading. The tissue chamber 20 height was 1.5 mm, slightly larger than minced tissue, to prevent clogging during sample loading and device 12 operation. Fluidic channels 28 were placed upstream and downstream of the tissue chamber 20, and in both cases, four channels that were 250 μm wide were employed. The symmetric channel 28 design was chosen for the minced format because there is a greater emphasis on prevention of clogging. The channel length was extended to 4 mm to prevent larger tissue pieces from squeezing all the way through, but flared the end to make it easier to connect with the underlying via layer.

The dissociation/filter device 40 processes tissue fragments and cell aggregates that are small enough to leave the tissue chamber 20 of the digestion device 12. This includes disaggregation via shear forces generated within the branching channel array (i.e., dissociation channels 52) and via physical interaction with nylon mesh filters 62, 64. Here, the dissociation and filter functionality has been integrated into a single device 40 to minimize holdup volume and simplify operation. The minced digestion and integrated dissociation/filter devices 12, 40 were fabricated using a commercial laminate process, with channel features laser micro-machined into hard plastic (PMMA or PET). All layers and other components were then aligned and bonded using pressure sensitive adhesive. Photographs of the fabricated devices are shown in FIGS. 2D and E.

Platform Optimization Using Murine Kidney

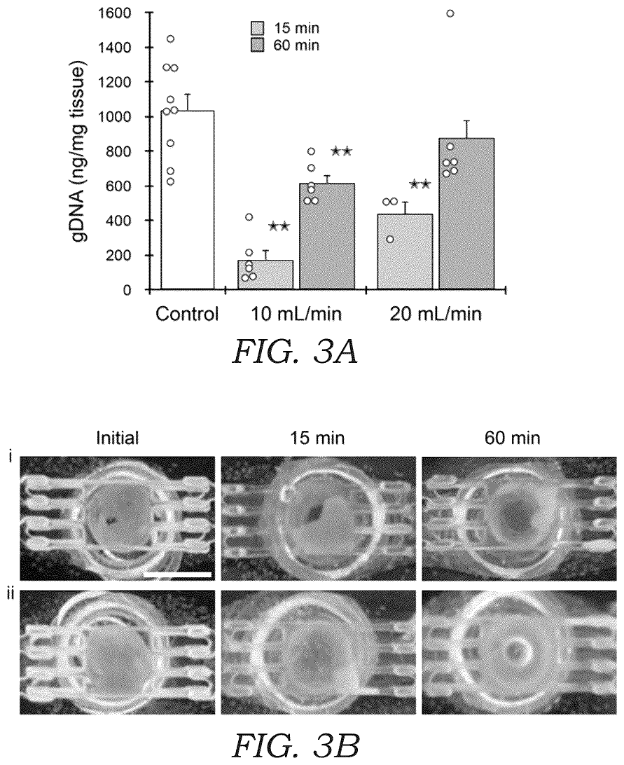

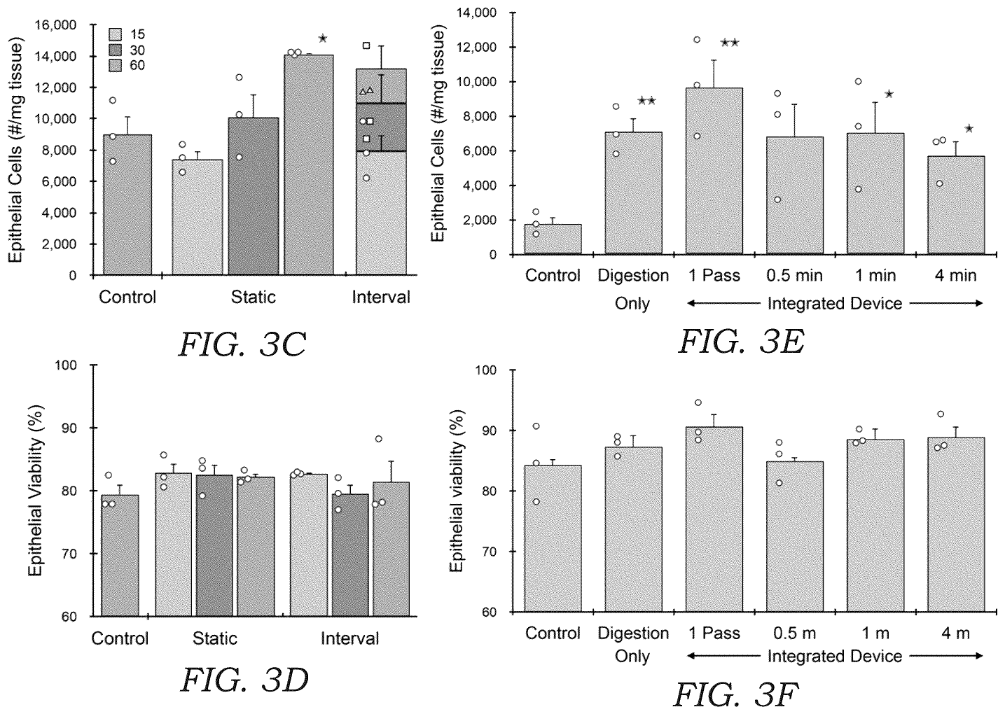

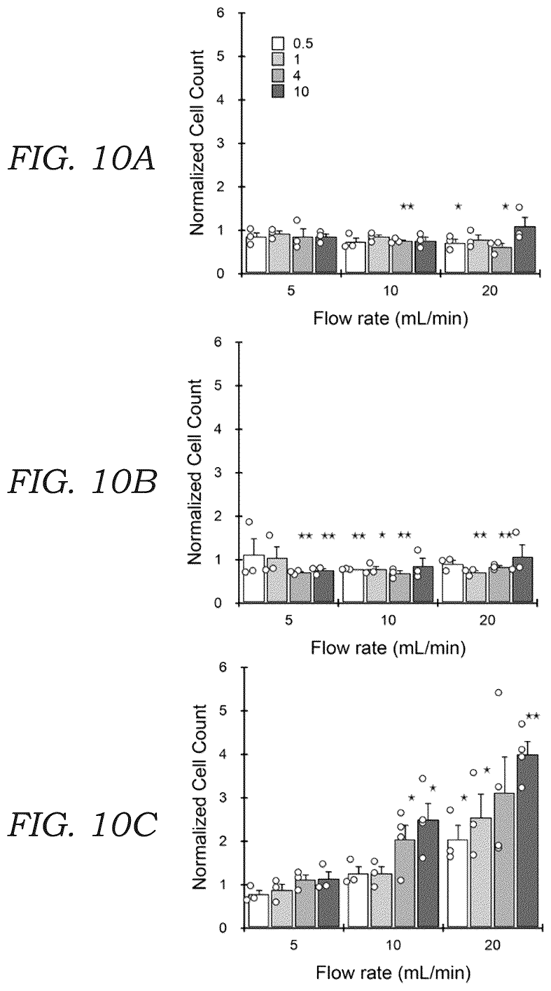

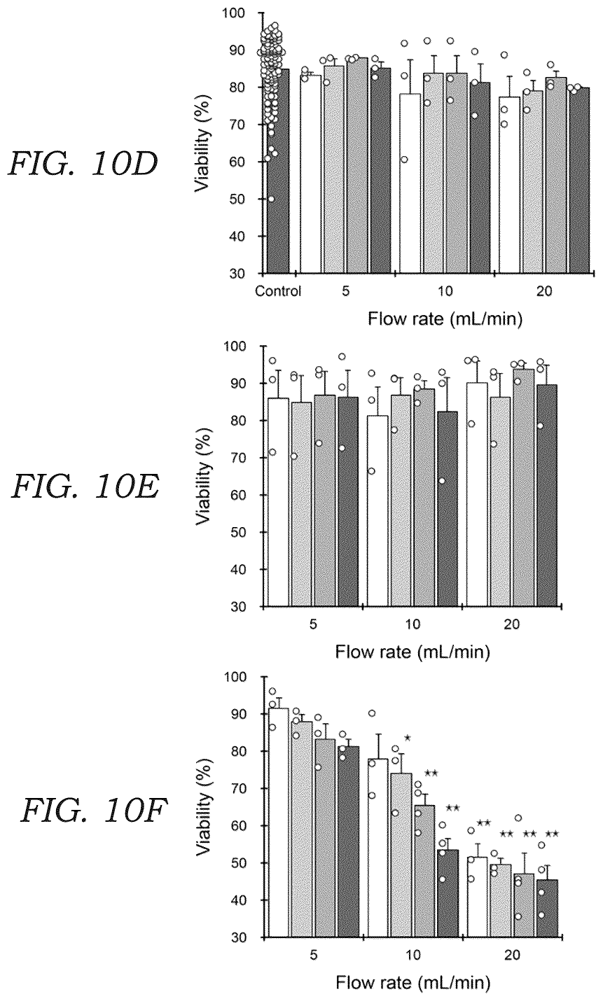

The digestion device 12 was evaluated using adult murine kidney samples. The kidney is a complex organ composed of anatomically and functionally distinct structures, and adult kidney tissue has a dense extracellular matrix that is challenging to dissociate into single cells. Freshly dissected kidneys were minced using a scalpel to ˜1 mm³ pieces and loaded into the minced digestion device 12 through the luer port 30. The device 12 and tubing 24 were then primed with PBS containing 0.25% type I collagenase, the luer input port 30 was sealed using a stopcock, and fluid was recirculated through the device 12 using a peristaltic pump 18. Flow rates of 10 and 20 mL/min were tested. After 15 or 60 min of recirculation, sample was collected, washed, and genomic DNA (gDNA) was extracted to assess total cell recovery. A control was minced and gDNA was directly extracted to provide an upper recovery limit. At 10 mL/min, gDNA was ˜15% and 60% of the control after 15 and 60 min, respectively (FIG. 3A). Increasing flow rate to 20 mL/min improved results to ˜40% and 85%, respectively. Images of the tissue chamber 20 were captured at the end of each experiment, and representative results are shown in FIG. 3B. Tissue was consistently observed to remain in the tissue chamber 20 or adjacent channels 28 at 10 mL/min, corroborating low gDNA recovery results. After 60 min at 20 mL/min, only a small amount of tissue was found within channels/vias, which helps explain why gDNA recovery was slightly lower than the control. Another possibility is that cells were damaged or destroyed during recirculation. To address this concern, MCF-7 breast cancer cells were recirculated through the system 10 and assessed cell number and viability (see FIGS. 10A-10F). It was observed that cell recovery decreased by ˜10% after recirculating through the digestion device 12, regardless of flow rate or time. Moreover, results were similar after recirculating through the peristaltic pump 18 alone, and cell viability remained high for all conditions tested. This confirms that sample loss was most likely related to hold-up within the system 10 or transfer steps, and not damage. Since 20 mL/min was more effective at clearing the tissue chamber 20 and isolating gDNA, it was used for the remainder of the experiments.

Next, single cells were analyzed using flow cytometry. Cell suspensions were labeled using a panel of antibodies and fluorescent probes specific for EpCAM (epithelial cells), TER119 (red blood cells), CD45 (leukocytes), and 7-AAD (live/dead), as listed in Table 1.

TABLE 1

| | | | Fluorophore | | | | | |
|---|---|---|---|---|---|---|---|---|
| | Antibody | Dilution | Kidney | Kidney | | | | |
| Assay | Clone | μg/mL | (Initial) | (Final) | Tumor | Liver | Heart | Positive Cells |
| EpCAM | G8.8 | 7 | PE | PE | PE | N/A | N/A | Epithelial cells |
| TER-119 | TER-119 | 5 | AF647 | AF647 | AF647 | AF647 | AF647 | Red blood cells |
| CD45 | 30-F11 | 5 | AF488 | BV510 | BV510 | BV510 | BV510 | Leukocytes |
| | | (AF488) | | | | | | |
| | | or 12.5 | | | | | | |
| | | (BV510) | | | | | | |
| Viability | N/A | 3.33 (7- | 7-AAD | 7-AAD | 7-AAD | 7-AAD | Zombie | Dead cells |
| | | AAD) or | | | | | Violet | |
| | | 1:1000 | | | | | | |
| | | (ZV) | | | | | | |
| CD31 | MEC13.3 | 8 | N/A | AF488 | AF488 | AF488 | AF488 | Endothelial cells |
| ASGPR1 | 8D7 | 10 | N/A | N/A | N/A | PE | N/A | Hepatocytes |
| Troponin T | REA400 | 0.15 | N/A | N/A | N/A | N/A | PE | Cardiomyocytes |

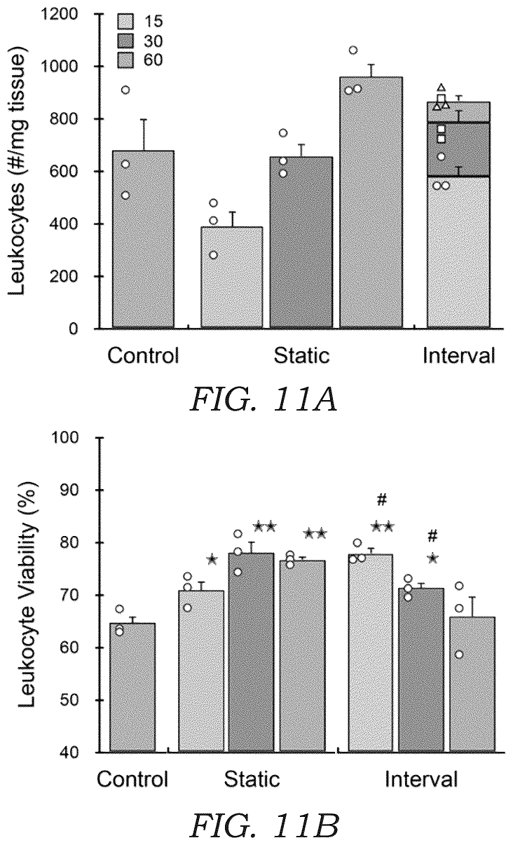

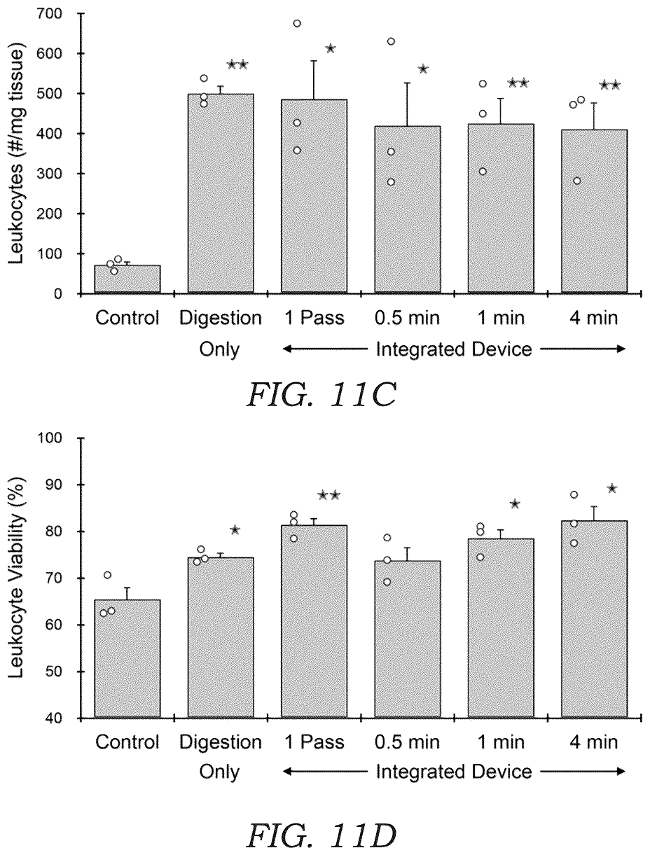

It was found that single epithelial cell numbers increased with processing time, from 15 to 60 min, producing up to ˜14,000 cells/mg tissue (FIG. 3C). This represents a 1.5-fold increase relative to the control, which was digested for 60 min under constant agitation, followed by repeated pipetting and vortexing to replicate standard tissue dissociation protocols. Note that after only 15 min in the digestion device 12, epithelial cells were statistically similar to the control digested for 4-fold longer time. An interval operation format was also investigated, which involved processing for short time periods, eluting the cell suspension, replacing collagenase in the digestion device 12, and continuing recirculation. It was observed that epithelial cell numbers accumulated through each time point of interval operation in a comparable manner to static operation. This demonstrates that interval collection does not compromise results, and suggests that epithelial cells can withstand long-term recirculation. Epithelial cell viability was ˜80% for all control and device conditions, further confirming that device processing did not adversely affect cells (FIG. 3D). Results in terms of cell number and viability were similar for leukocytes (see FIGS. 11A and 11B).

The integrated dissociation/filter device 40 was then investigated if it could further enhance single cell yield following the digestion device 12 (FIGS. 10A-10F). Initial tests were performed using the MCF-7 model, and it was found that recirculation at 20 mL/min, even for short periods of time, resulted in low viability. At 10 mL/min, single cells increased by ˜20% after 30 s of recirculation, with no change in viability. Longer recirculation times enhanced single cell numbers but decreased viability. Thus, short recirculation times at 10 mL/min using minced kidney that had been processed using the digestion device for 15 min was selected. As a final step, sample was passed through the nylon mesh membranes in the filters 62, 64 at 10 mL/min. Single epithelial cell recovery numbers are presented in FIG. 3E. The digestion device 12 produced 4-fold more single cells than the control that was also digested for 15 min. A single pass through the integrated device (digestion device 2 and dissociation/filter device 40) increased single epithelial cells by ˜40% compared to digestion alone, which was ˜5.5-fold greater than the control. Recirculation through the branching channel array produced fewer cells than the single pass. Epithelial cell viability was ˜85-90% for all conditions (FIG. 3F). Similar results were observed for leukocytes (See FIGS. 11A-11D). Based on these results, a single pass operation was selected through the integrated dissociation/filtration device 40 for all work with kidney. Note that the integrated device obviates the need for a cell straining step prior to flow cytometry.

Single Cell Analysis of Murine Kidney

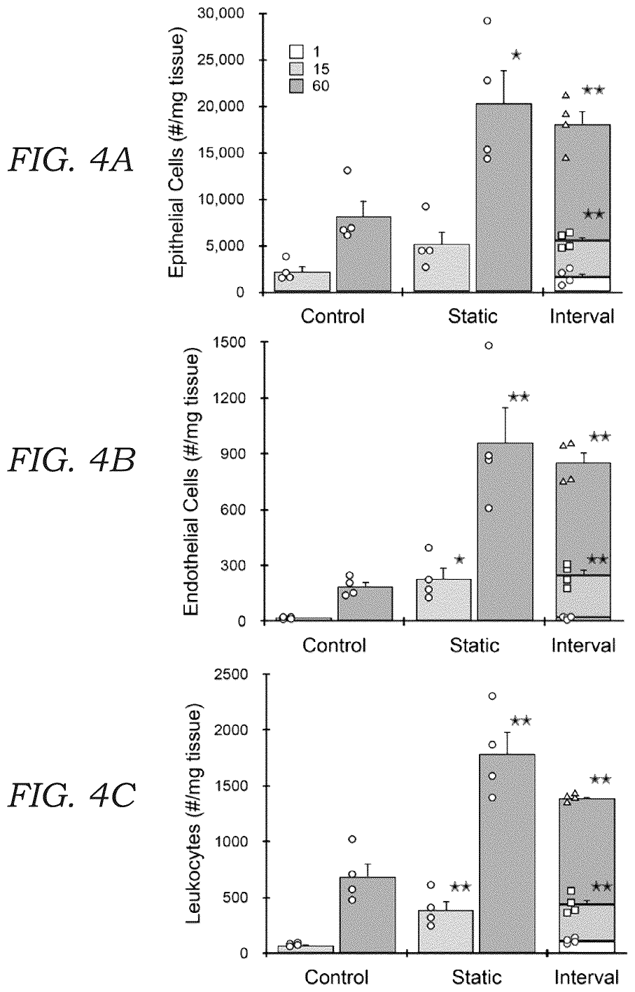

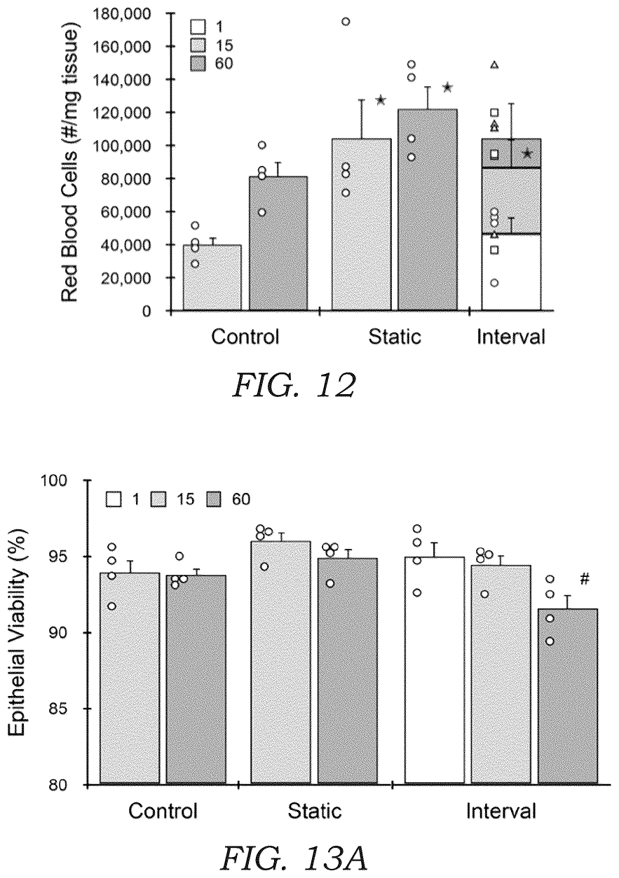

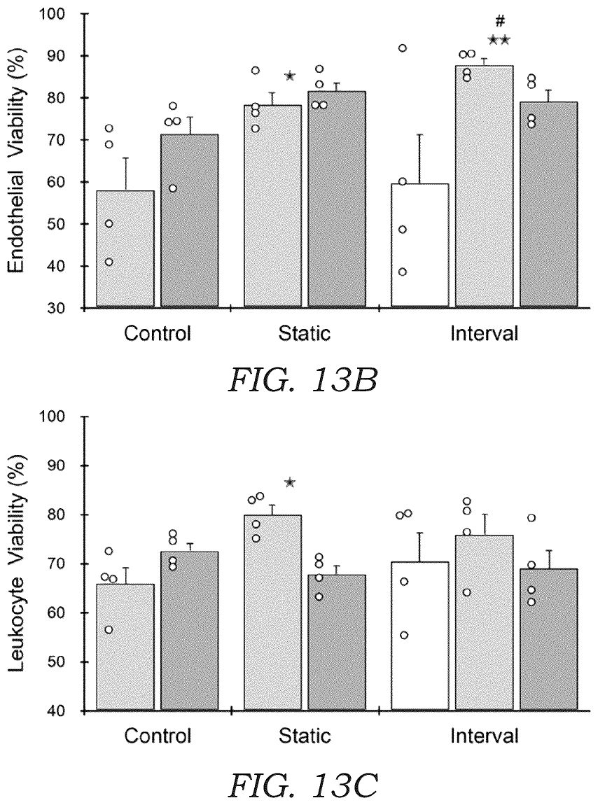

Kidney was evaluated under different digestion times using the full microfluidic platform. Endothelial cells (via CD31, Table 1) were also added to the flow cytometry panel, since they are difficult to isolate using traditional dissociation methods. Minced tissue was loaded into the digestion device 12 and processed under static (15 or 60 min) or interval (1, 15, and 60 min) formats, and then passed through the integrated dissociation/filter device 40 one time. Controls were minced, digested (15 or 60 min), disaggregated by vortexing/pipetting, and filtered using a cell strainer. Results for epithelial cells are presented in FIG. 4A, and are generally similar to optimization studies (FIG. 3C), although epithelial cells increased to ˜20,000/mg tissue. This was ˜40% higher than the optimization study due to the integrated dissociation/filter 40, and overall more than double the 60 min control. Surprisingly, the 1 min interval produced ˜1500 epithelial cells/mg, which was similar to the 15 min control. This time point was chosen primarily to eliminate erythrocytes (see FIG. 12). Device processing was even more effective for endothelial cells (FIG. 4B), which exceeded the 60 min control by >5-fold. Findings for leukocytes (FIG. 4C) were generally similar to epithelial cells. A slight decrease in total cell recovery was observed for the interval format relative to the 60 min static condition for all cell types, although this was not statistically significant. This modest decrease may have been due to sample loss during transfer and/or priming steps. Alternatively, cell clusters may have eluted in the early intervals, which would have otherwise been broken down if they remained within the digestion device. Relative to the 60 min control, endothelial cells were enriched for all device conditions except the 1 min interval. Leukocytes were present at similar levels except in the 15 min control, where they were under-represented. Interestingly, batch-to-batch reproducibility, as measured by the coefficient of variance (see Table 2 below), decreased with processing time for each condition, and was lowest for the microfluidic system 1 using intervals. Viability for all three cell types after device processing were similar to or exceeded controls (see FIGS. 13A-13C).

TABLE 2

| Condition | Epithelial Cells | Endothelial Cells | Leukocytes |
|---|---|---|---|
| Control 15 m | 24.5 | 15.4 | 10.4 |
| Control 60 m | 20.2 | 13.2 | 17.3 |
| Static 15 m | 27.2 | 25.3 | 20.3 |
| Static 60 m | 17.1 | 19.2 | 11.1 |
| Interval 1 m | 25.4 | 18.5 | 11.3 |
| Interval 15 m | 7.5 | 11.7 | 10.2 |
| Interval 60 m | 7.8 | 6.5 | 1.2 |

Table 2 shows the coefficient of variation values for kidney samples at different processing conditions.

Next scRNA-seq was performed, which has been used to catalogue the diverse cell types residing within murine kidney and create atlases. Kidney tissue was processed using the system 10 and collected at 15- and 60-min intervals along with evaluation of the 60 min control. Live single cells were isolated from debris and dead cells using fluorescence-activated cell sorting (FACS), loaded onto a droplet-enabled 10× Chromium platform, and 34,034 cells were sequenced at an average depth of ˜60,000 reads/cell. scRNA-seq quality metrics are shown in Table 3 below, and were comparable across conditions.

TABLE 3

| | | | | | | Reads Mapped Confidently | | | |
|---|---|---|---|---|---|---|---|---|---|
| | | Mean | | | | to | | | Fraction |
| | | Reads/ | Mean | Mean | Total | | Exonic | | Reads |
| Cond. | Tissue | Cell | UMI | Gene | Gene | Genome | Regions | Transcriptome | in Cell |
| 60 | Kidney | 61626 | 6091 | 1818 | 20138 | 93.4% | 78.00% | 75.1% | 80.6% |
| Control | | | | | | | | | |
| 15 | Kidney | 57453 | 8244 | 2076 | 19761 | 93.6% | 78.5% | 75.70% | 91.4% |
| Platform | | | | | | | | | |
| 60 | Kidney | 59596 | 5575 | 1770 | 21487 | 93.0% | 74.7% | 71.60% | 80.9% |
| Platform | | | | | | | | | |
| 60 | Breast | 44440 | 8412 | 2335 | 20908 | 90.5% | 68.5% | 65.5% | 92.7% |
| Control | Tumor | | | | | | | | |
| 15 | Breast | 41371 | 10286 | 2836 | 20895 | 90.4% | 68.3% | 65.0% | 94.2% |
| Platform | Tumor | | | | | | | | |
| 60 | Breast | 47196 | 10788 | 2677 | 21357 | 91.1% | 69.8% | 66.8% | 95.5% |
| Platform | Tumor | | | | | | | | |

Table 3 shows scRNA-seq metrics for kidney and breast tumor samples.

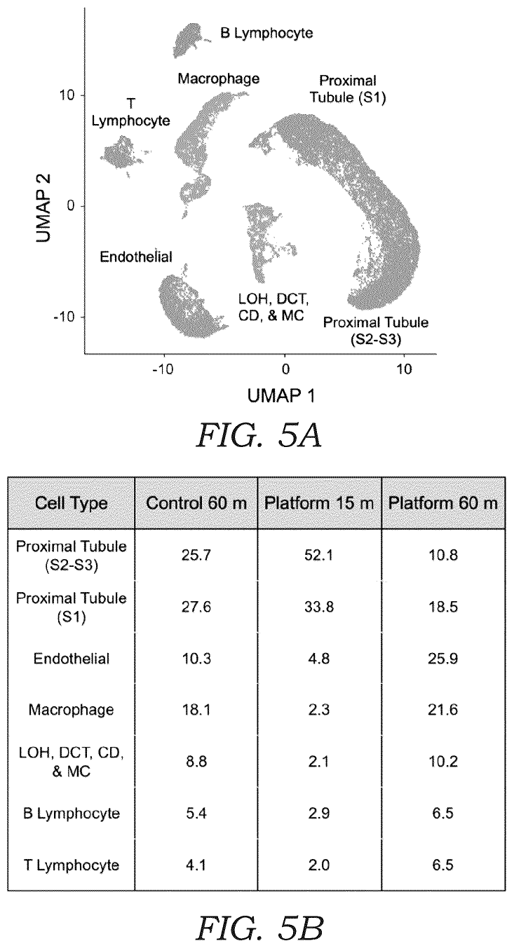

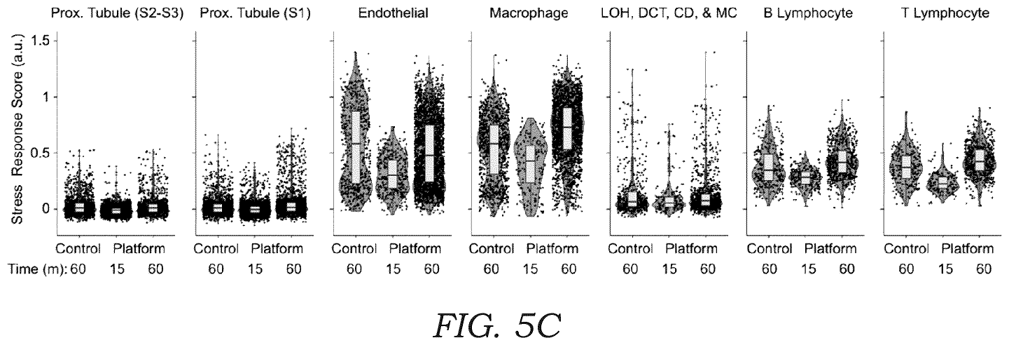

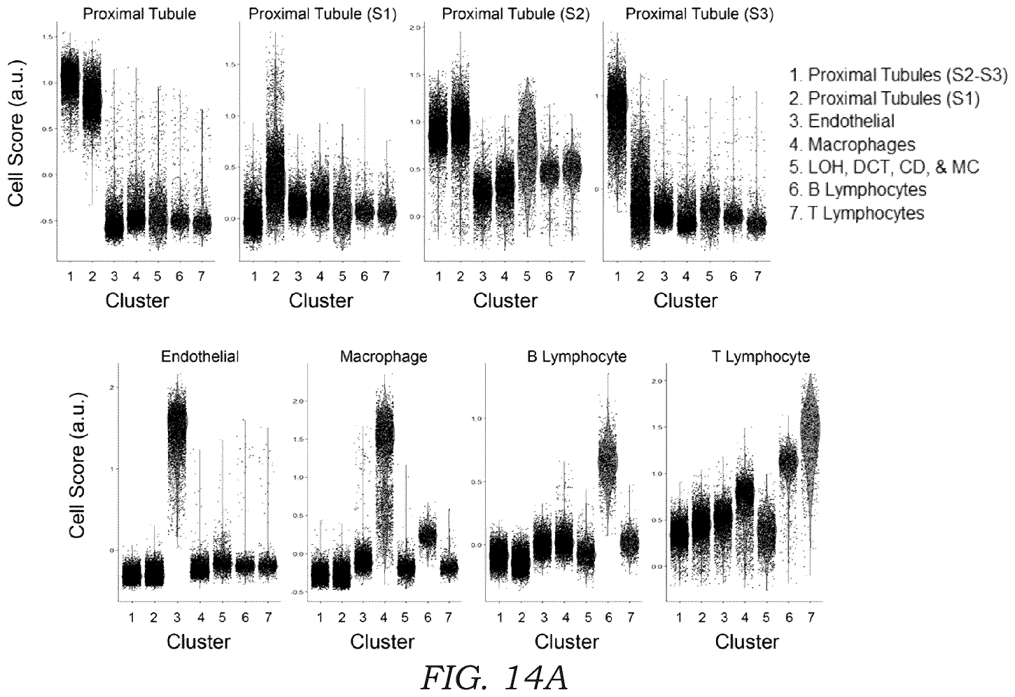

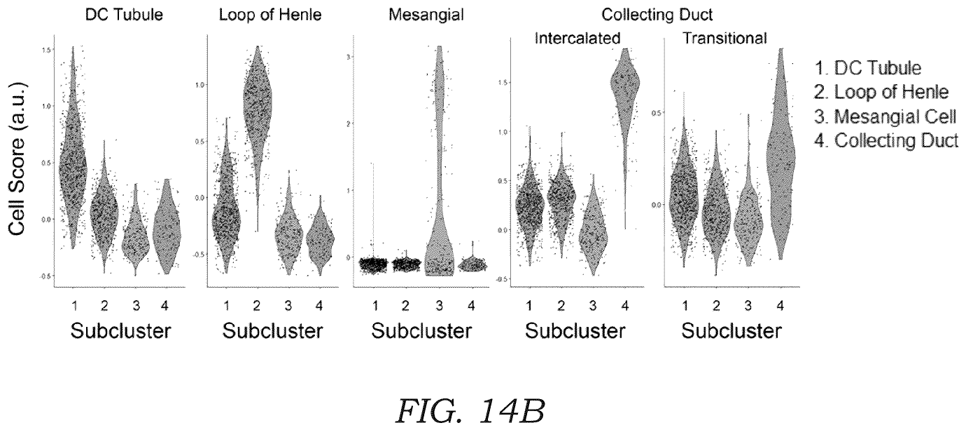

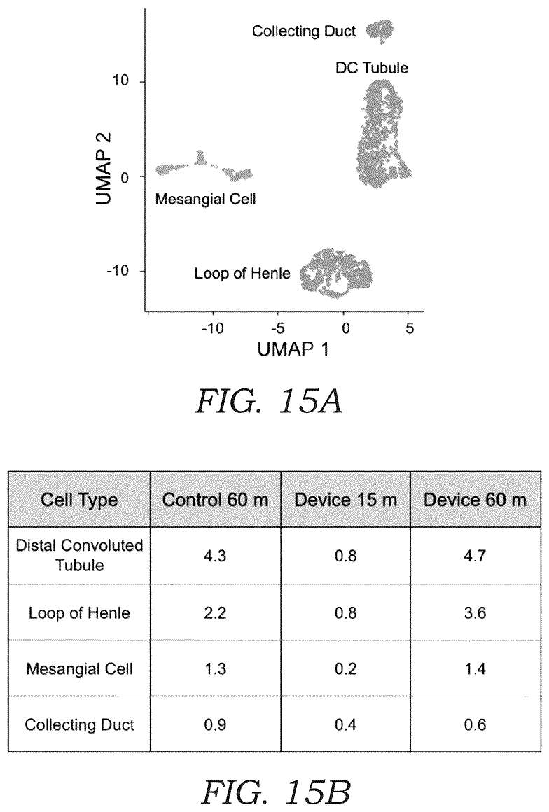

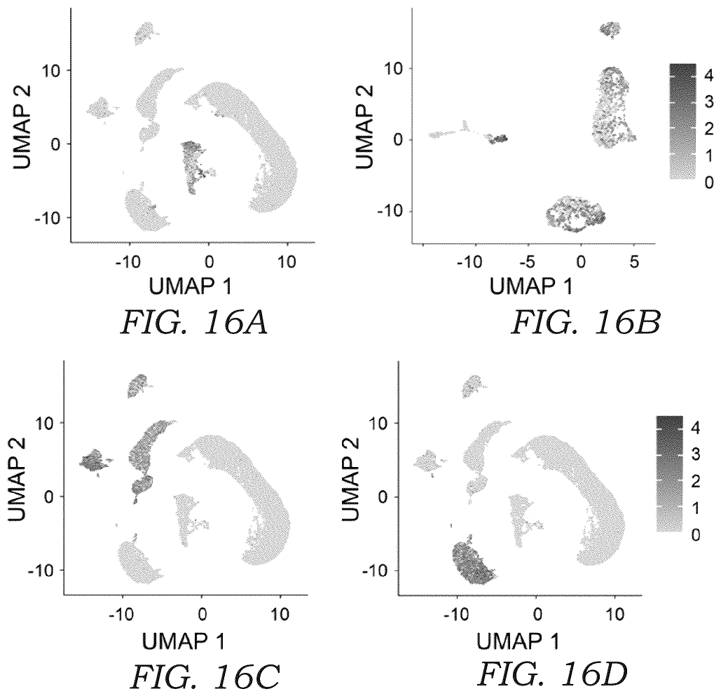

After filtering, Seurat was used to identify (FIG. 5A) and annotate (see FIGS. 14A-14B) seven cell clusters. This included two clusters of proximal tubules (convoluted, or S1, and straight, or S2-S3), endothelial cells, macrophages, B lymphocytes, and T lymphocytes. The final cluster was heterogenous, and included cells from the distal convoluted tubule (DCT), Loop of Henle (LOH), and collecting duct (CD), as well as mesangial cells (MC). All seven clusters were represented in control and device conditions. The relative number of cells in each cluster are shown in FIG. 5B. Proximal tubules were the predominant cell population, representing ˜53% of the control, which closely matched a recently published mouse kidney atlas. Proximal tubules were further enriched in the 15 min device condition, comprising ˜86% of the cell suspension. The other cell populations were under-represented relative to the control, most by ˜2-fold, but reaching as high as 8-fold for macrophages. However, it is unclear whether this was caused by diminished recovery or simply dilution by proximal tubules. The 60 min device interval only contained ˜29% proximal tubules, but it was surmised that most had already been removed in the 15 min interval. Endothelial cells were clearly enriched at 60 min, increasing to ˜25% of the suspension, while remaining cell types remained close to control values. Similar trends were observed within the DCT, LOH, DC, and MC sub-clusters (see FIGS. 15A, 15B). To compare population percentages obtained from scRNA-seq (FIG. 5B) and flow cytometry, consideration must be given to which cell populations were likely to express each marker. CD45 and CD31 gene expression was well correlated with the appropriate clusters (see FIGS. 16A-16D). For EpCAM, DCT and CD cells have been shown to express at high levels, while proximal tubules and LOH cells ranged from low to undetectable. Inspection of sequencing results indicated that EpCAM was highly expressed by at least a subset of the DCT, LOH, CD, & MC sub-clusters (see FIGS. 16A-16D). Interestingly, proximal tubules were predominantly EpCAM-negative, but this could be explained by low basal expression and/or a potential secondary factor such as low protein turnover. The brightest fluorophore was used to stain EpCAM, phycoerithrin (PE), to help discern low level expression, but it is possible that some cell proximal tubules remained undetectable. Assuming all proximal tubule, DCT, LOH, CD, & MC clusters were EpCAM+, the calculated population percentages were ˜62, 88, and 40% for the control, 15 m device, and 60 m device conditions, respectively. This is directly in line with flow cytometry results for the 15 m device case, but considerably lower for the others. It should be noted that if flow cytometry missed any of these cell types due to low EpCAM expression, it would only widen the disparity. Instead, it is proposed that the comprehensive manner in which scRNA-seq identifies cell types is superior to flow cytometry, particularly when a clear positive biomarker for all cell sub-populations is lacking. Flow cytometry is better suited to cell counting, however, and based on those results, device processing consistently produced comparable numbers of cells at 15 min and at least 50% more cells at 60 min, relative to the 60 min control. These estimates were used as weighting factors (1× for 15 min, 1.5× for 60 min), along with percentages in FIG. 5B, to calculate aggregate device platform yields (see Table 4).

TABLE 4

| | | | Device | |
|---|---|---|---|---|
| | Device | Device | Total | Device |
| | 60 m | Total | (Norm. to | Total |
| Cluster | (weighted) | (weighted) | control) | (%) |
| Proximal Tubule | 16.2 | 68.3 | 2.7 | 27.3 |
| (S2-S3) | | | | |
| Proximal Tubule | 27.8 | 61.6 | 2.3 | 24.6 |
| (S1) | | | | |
| Endothelial | 38.9 | 43.7 | 4.2 | 17.5 |
| Macrophage | 32.4 | 34.7 | 1.9 | 13.9 |
| LOH, DCT, | 15.3 | 17.4 | 2.0 | 7.0 |
| CD, & MC | | | | |
| LOH | 7.0 | 7.9 | 1.8 | 3.1 |
| DCT | 5.4 | 6.2 | 2.8 | 2.5 |
| CD | 2.1 | 2.3 | 1.8 | 0.9 |
| MC | 0.9 | 1.3 | 1.4 | 0.5 |
| B Lymphocyte | 9.8 | 12.7 | 2.3 | 5.1 |
| T Lymphocyte | 9.8 | 11.8 | 2.9 | 4.7 |

Table 4. Weighted population values for each cluster and sub-cluster in murine kidney. Population percentages for microfluidic processing in FIG. 5B were weighted (1× for 15 min and 1.5× for 60 min) and added to create total aggregate microfluidic platform values. These were normalized by the control and used to calculate total aggregate population distributions.

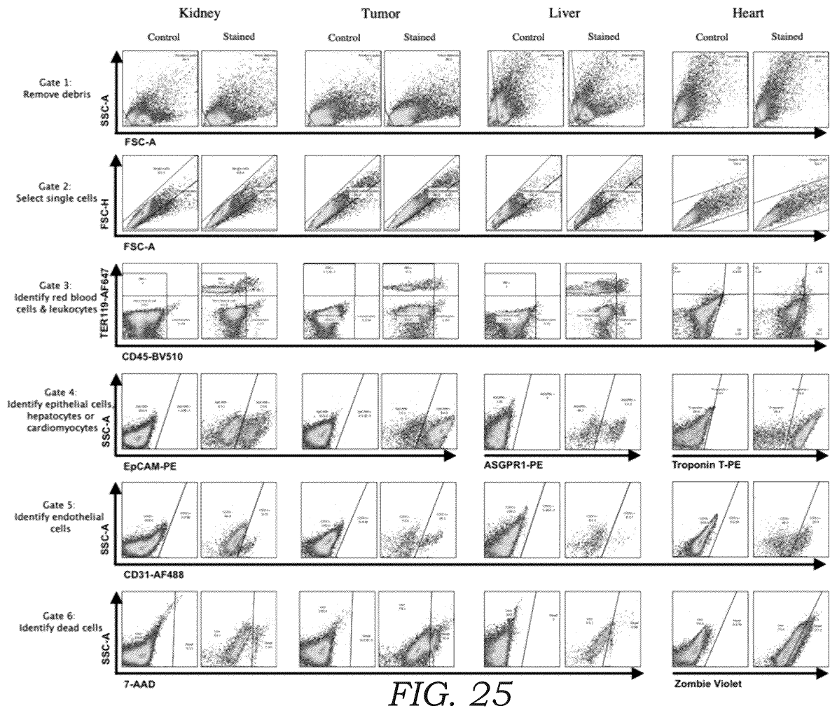

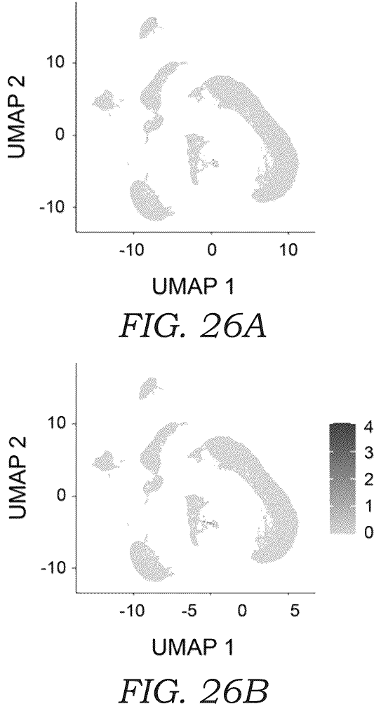

Total endothelial cell recovery was ˜4-fold greater than the control, while other cell types were ˜2- to 2.5-fold greater, which all match flow cytometry (FIGS. 4A-4C). While the true weighing factors may be slightly different, it does appear that the relative numbers between control and device platform are consistent between flow cytometry and scRNA-seq. However, the relative numbers across cell types varies considerably, which may have resulted from biasing during FACS collection or droplet loading in the 10× Chromium system, which have been documented previously. The results suggest a preferential selection of endothelial cells and leukocytes during these steps. Nevertheless, the microfluidic system 10 can address cell-specific biasing of kidney tissue during isolation by enriching endothelial cells, which have been shown to be underrepresented using standard tissue dissociation workflows, while maintaining all other cell subtypes at similar numbers. It is notable that only a few potential podocytes were observed in either control or device samples (see FIGS. 26A-26B), which may be attributed to the fact that collagenase was used for enzymatic digestion. Kidney atlases prepared using Liberase also lacked podocytes, while the combination of collagenase and Pronase, as well as a cold-active protease, yielded podocyte cell clusters. This indicates that the choice of enzyme is still important even in settings with enhanced mechanical forces.

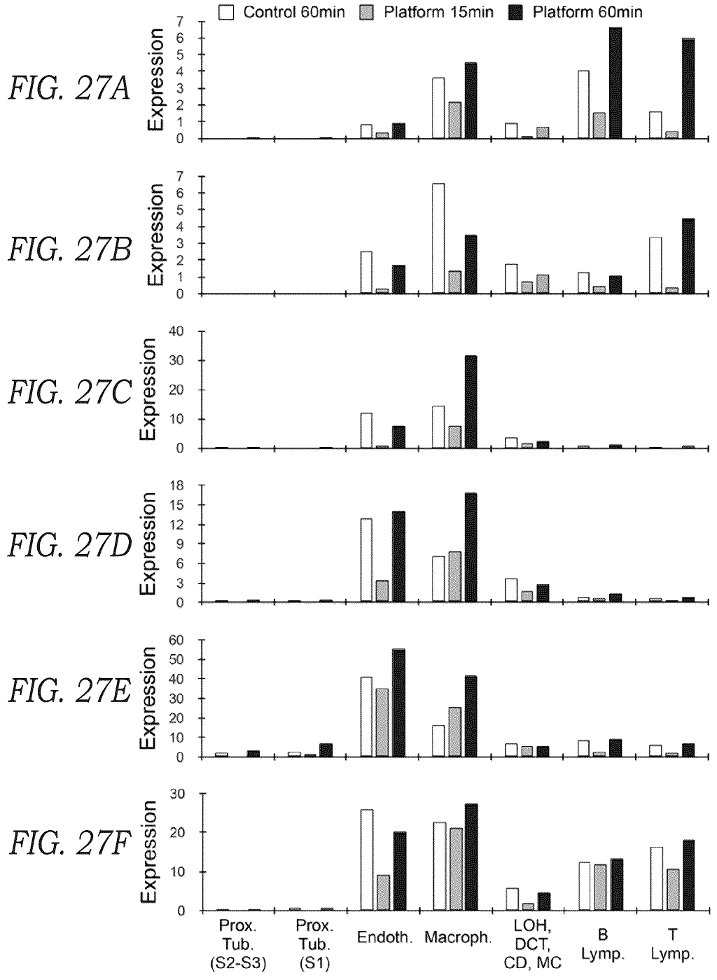

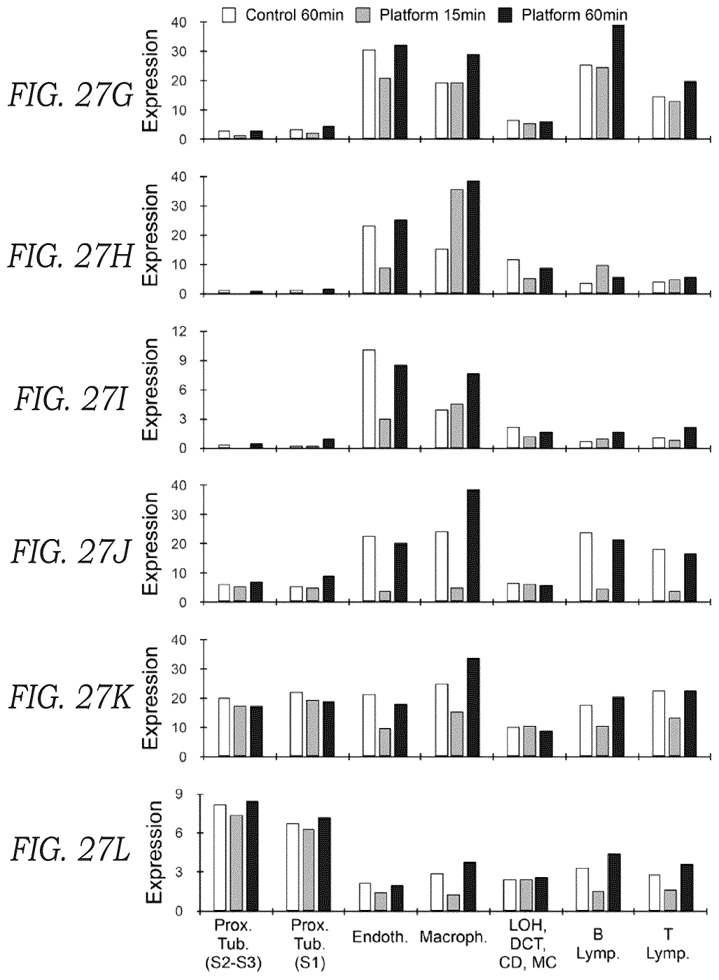

Lastly, stress response genes were evaluated, which can interfere with cell identification using transcriptomic information. Induction of stress responses have been linked to conventional tissue dissociation protocols. Since a large number of genes have been implicated, a stress response score is calculated based on previous scRNA-seq work, and results are presented in FIG. 5C. It was found that stress response scores were cell type specific, with proximal tubules exhibiting the lowest values, as recently reported. Stress response scores were generally lower for the 15 min interval condition compared to the 60 min interval and control cases. This is consistent with previous findings that shortening enzymatic digestion time reduces dissociation-induced transcriptional artifacts. Importantly, no evidence was found that exposure to fluid shear stresses within the digestion device heightened the stress response for any cell type. This suggests that time was the predominant factor, which can be mitigated using the interval concept in the microfluidic platform. Expression values for selected stress response genes are individually shown in FIGS. 27A-27C.

Processing and Single Cell Analysis of Murine Breast Tumor Tissue

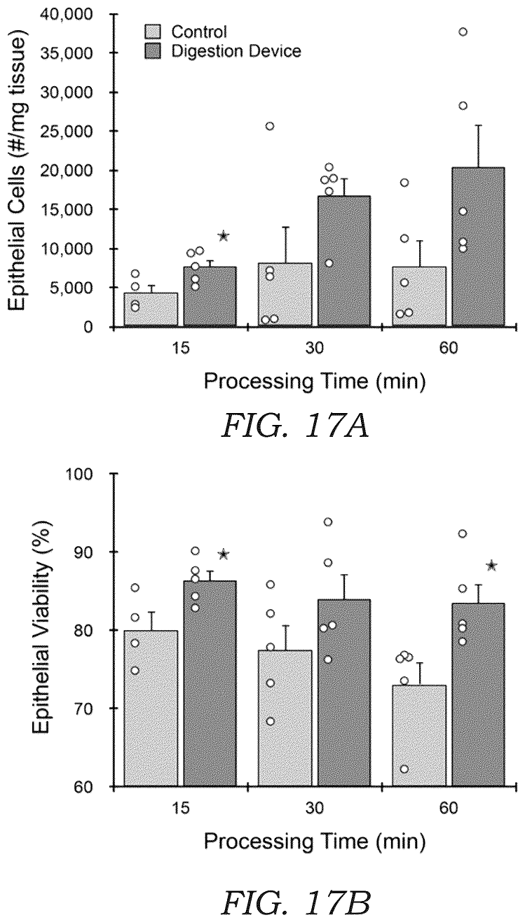

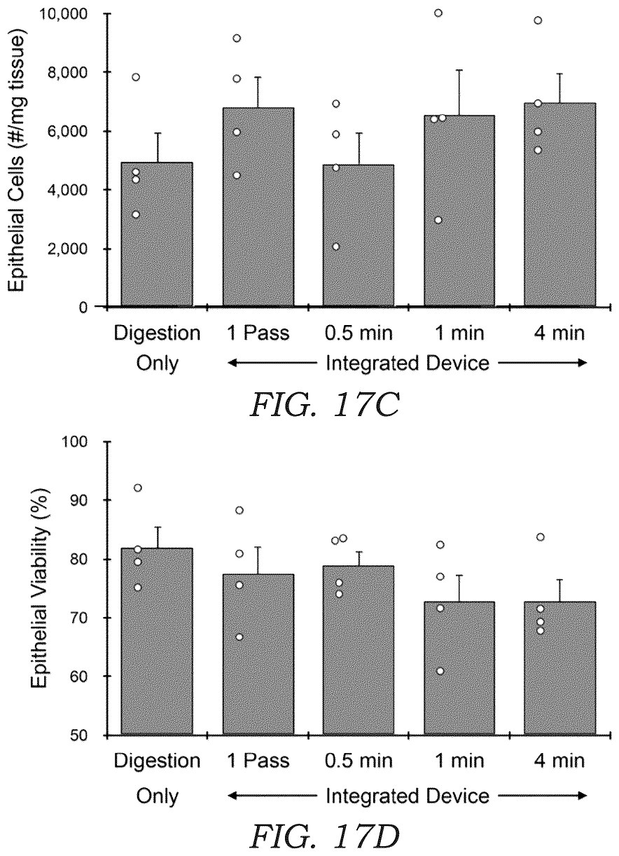

Solid tumors can exhibit high degrees of intratumoral heterogeneity, which has been directly implicated in cancer progression, metastasis, and the development of drug resistance. This heterogeneity has successfully been captured using scRNA-seq and linked to survival for glioblastoma, drug resistance in melanoma, and prognosis for colorectal cancer. Moreover, it is expected that expanded application of scRNA-seq in clinical settings will soon emerge to provide molecular and cellular information for guiding personalized therapies. Due to abnormal extracellular matrix composition and density, however, tumor tissues are considered to be amongst the most difficult epithelial tissues to dissociate. Microfluidic processing of mammary tumors that spontaneously arise in MMTV-PyMT transgenic mice was evaluated. First, the minced digestion device 12 and integrated dissociation/filter device 40 were optimized separately. The digestion device 12 generated ˜2-fold more EpCAM+ epithelial cells than the controls after 15 and 30 min, and the difference extended to 2.5-fold after 60 min (see FIG. 17A). Viability was higher for device processed samples than controls at all time points (see FIG. 17B). Next, the integrated dissociation/filter device 40 was tested and again found that a single pass was optimal (see FIG. 17C, 18D). In this case, recirculation for 1 and 4 min produced similar cell numbers, but with lower viability.

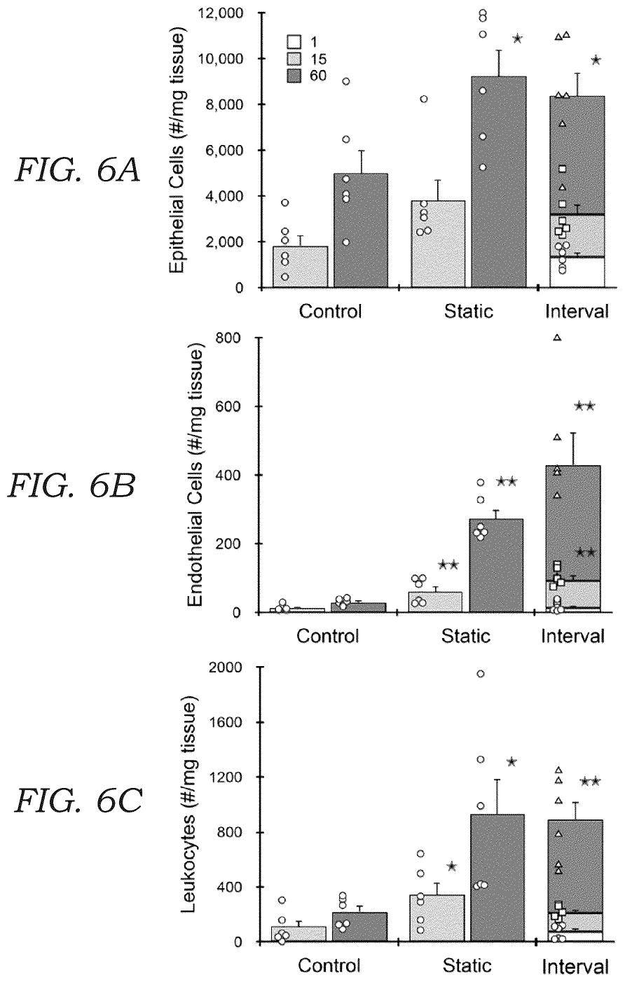

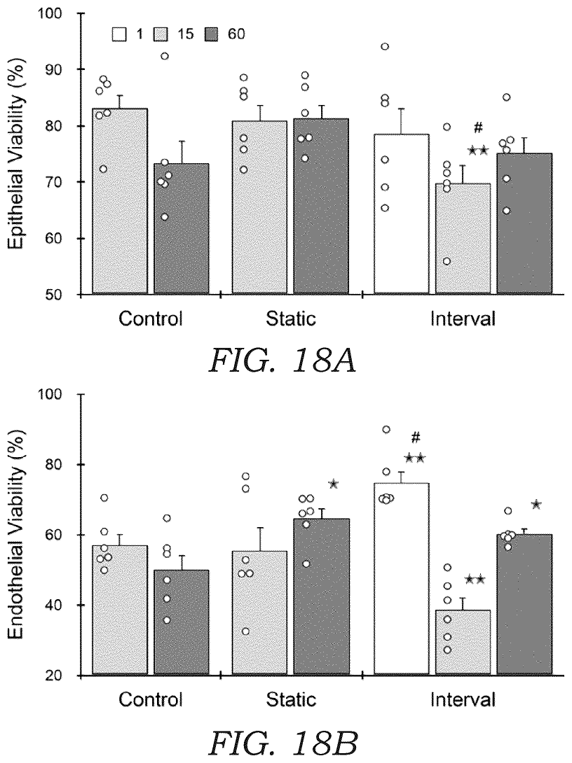

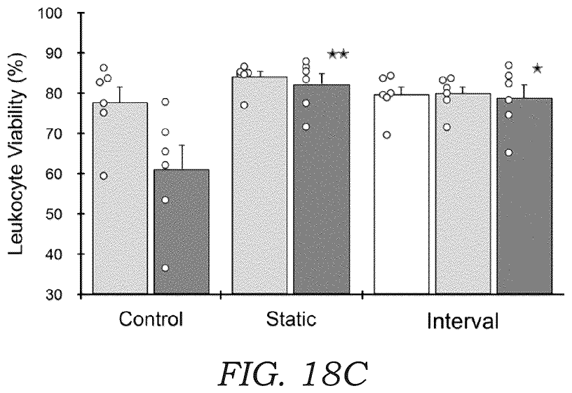

Results for the full microfluidic device system 10 are shown in FIGS. 6A-6C, and were generally similar to kidney, but with 2- to 3-fold lower cell counts/mg tissue. However, the system 10 still produced significantly more cells than controls. Epithelial cells were ˜2-fold higher at both time points (FIG. 6A). Endothelial cells were again liberated more effectively by device processing, with 5-fold more cells recovered after 15 min and 10-fold more after 60 min (FIG. 6B). Leukocytes increased by 3- and 5-fold after 15 and 60 min, respectively (FIG. 6C). The interval format produced similar total epithelial cell and leukocyte numbers when compared to the corresponding static time point. However, ˜30% more endothelial cells were obtained from intervals. It should be noted that a remarkably large number of epithelial cells (>15%) were obtained at the 1 min interval. Device processing enriched for endothelial cells and leukocytes at all but the 1 min time point, which remained similar to controls. As with kidney, microfluidic processing was associated with higher batch-to-batch reproducibility, as measured by the coefficient of variation (see Table 5 below). Viability for all three cell types were similar to the 15 min control and exceeded the 60 min control (see FIGS. 18A-18C). Thus, the microfluidic system 10 liberated more single cells from tumor, while also better preserving cell viability.

TABLE 5

| Condition | Epithelial Cells | Endothelial Cells | Leukocytes |
|---|---|---|---|
| Control 15 m | 25.4 | 26.4 | 41.0 |
| Control 60 m | 19.9 | 13.1 | 19.5 |
| Static 15 m | 23.5 | 23.7 | 24.6 |
| Static 60 m | 12.6 | 9.7 | 27.9 |
| Interval 1 m | 14.2 | 37.2 | 25.9 |
| Interval 15 m | 14.0 | 17.0 | 11.7 |
| Interval 60 m | 12.2 | 21.6 | 14.3 |

Table 5 shows the coefficient of variation values for breast tumor samples at different processing conditions.

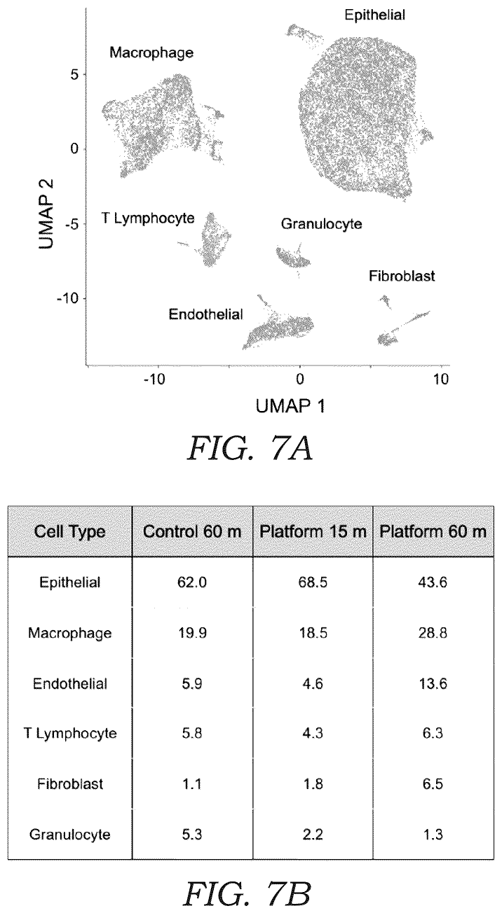

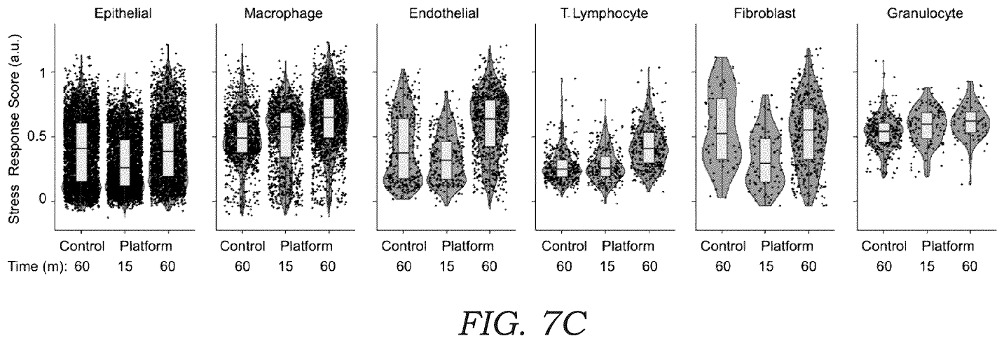

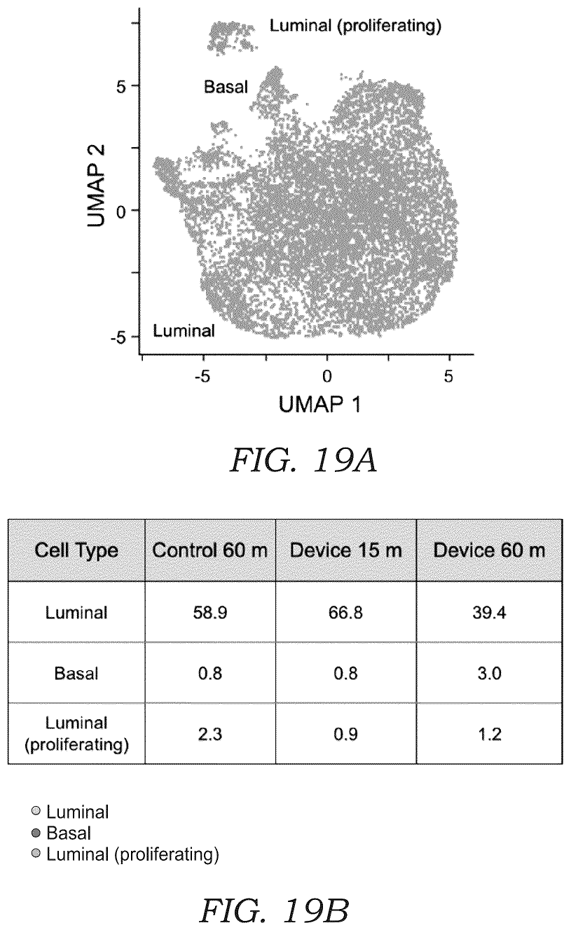

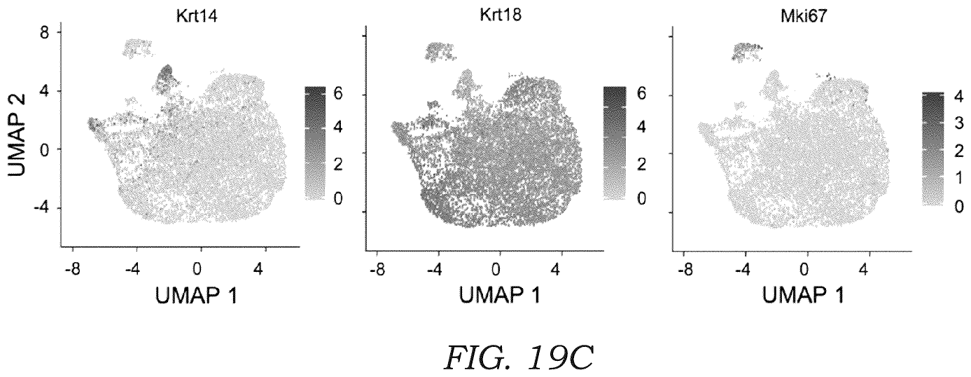

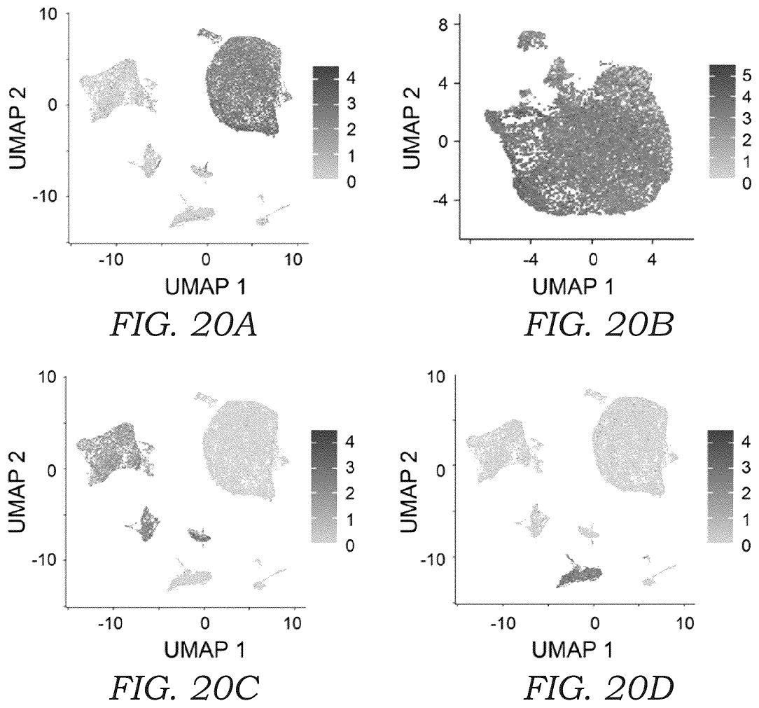

scRNA-seq was performed again using the 15- and 60-min device intervals and the 60 min control. A total 24,527 cells were sequenced at an average of ˜45,000 reads per cell. 6 clusters were identified corresponding to epithelial cells, macrophages, endothelial cells, T lymphocytes, fibroblasts, and granulocytes (FIG. 7A). Epithelial cells were the predominant cell population, representing 62.0% of control cells (FIG. 7B). Epithelial percentages increased slightly in the 15 min interval and decreased in the 60 min interval. Three sub-clusters were identified within the epithelial population corresponding to luminal, basal, and proliferating luminal cells based on expression of Krt14, Krt18, and Mki67, respectively (see FIGS. 19A-19C). The luminal sub-type dominated, as expected for MMTV-PyMT tumors. The basal subpopulation was enriched with device processing, while the proliferating luminal subpopulation was under-represented. These results suggest that basal epithelium is more difficult to dissociate. Comparing cell populations between scRNA-seq and flow cytometry was more straightforward since EpCAM, CD45, and CD31 were all correlated well with the expected cell types (see FIGS. 20A-20D). However, fibroblasts were not detected by flow cytometry, and account for a significant portion of the 60 min device condition. As with kidney, tumor epithelial percentages were significantly higher in flow cytometry data, which would further suggest biasing during sorting and/or droplet encapsulation. If one combines the population percentages in FIG. 7B with the same weighting factors used for kidney (lx for 15 min, 1.5× for 60 min), one can again calculate aggregate device platform yields (see Table 6).

TABLE 6

| | | | Device | |
|---|---|---|---|---|
| | Device | Device | Total | Device |
| | 60 m | Total | (Norm. to | Total |
| Cluster | (weighted) | (weighted) | control) | (%) |
| Epithelial | 65.4 | 133.9 | 2.2 | 53.5 |
| Luminal | 59.1 | 125.9 | 2.1 | 50.4 |
| Basal | 4.5 | 5.3 | 6.6 | 2.1 |
| Lum. Prolif. | 1.8 | 2.7 | 1.2 | 1.1 |
| Macrophage | 43.2 | 61.7 | 3.1 | 24.7 |
| Endothelial | 20.4 | 25.0 | 4.2 | 10.0 |
| T Lymphocyte | 9.5 | 13.8 | 2.4 | 5.5 |
| Fibroblast | 9.8 | 11.6 | 10.5 | 4.6 |
| Granulocyte | 2.0 | 4.2 | 0.8 | 1.7 |

Table 6 shows the weighted population values for each cluster and sub-cluster in murine breast tumor. Population percentages for microfluidic processing in were weighted (1× for 15 min and 1.5× for 60 min) and added to create total aggregate microfluidic platform values. These were normalized by the control and used to calculate total aggregate population distributions.

Differences for the device aggregate relative to the control were ˜2-fold for epithelial cells and 2.5- to 3-fold for T lymphocytes and macrophages, which are all similar to flow cytometry results (FIGS. 6A and 6C). Endothelial cells were ˜4-fold greater for the microfluidic system 10, which is lower than the 10-fold difference from flow symmetry (FIG. 6B). Notably, fibroblasts were enriched by 10-fold using the device platform. The results confirm that tissue processing with the microfluidic system 10 can improve isolation of all cell types by at least 2.5-fold, as well as difficult to liberate cell types such as endothelial cells, fibroblasts, and basal epithelium by 4- to 10-fold.

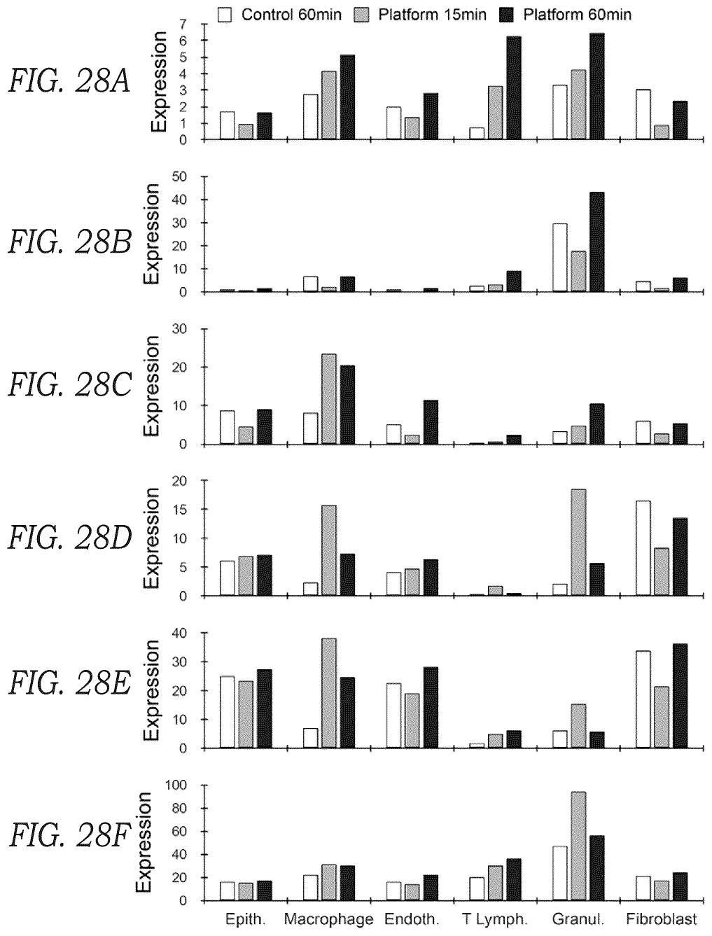

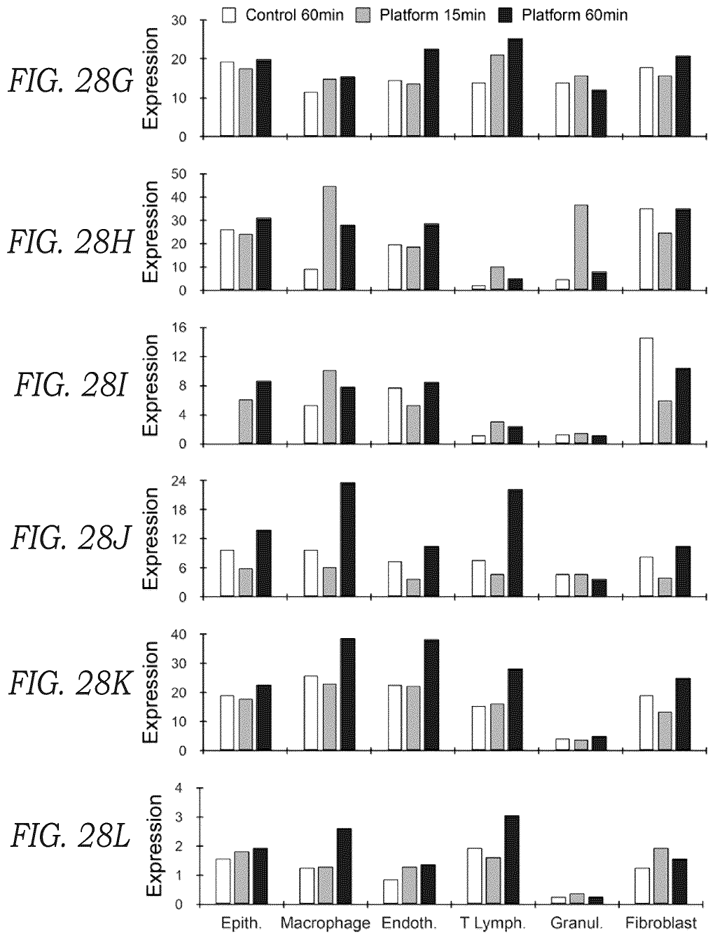

Finally, stress response scores were determined as described for kidney. The importance of stress responses can be heightened for tumor since some response genes, such as members of the Jun and Fos families, have been associated with metastatic progression and drug resistance. Stress response scores were similar across all cell types and conditions for tumor (FIG. 7C). It is possible that tumor cells are more sensitive to dissociation-induced transcriptional changes, and that even shorter intervals would be necessary to lower these responses. Expression values for selected stress response genes are individually shown in FIGS. 28A-28L.

Isolation of Hepatocytes from Murine Liver

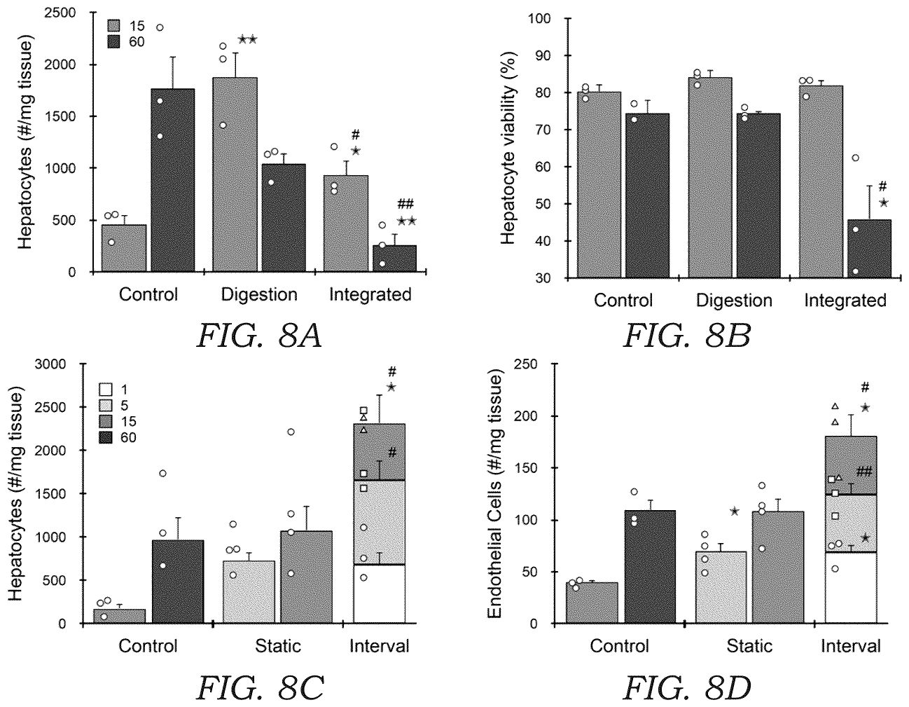

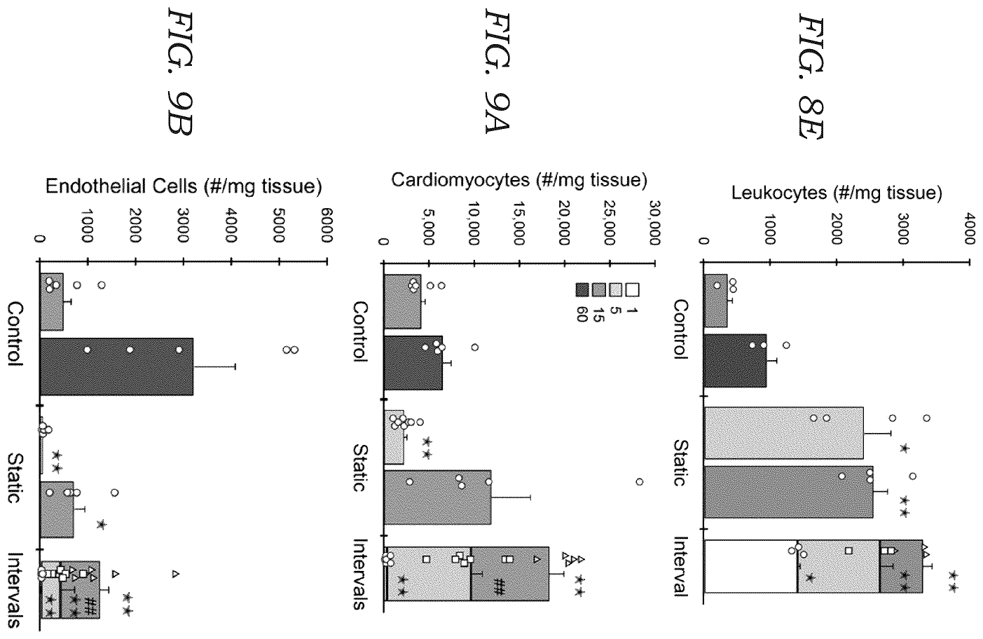

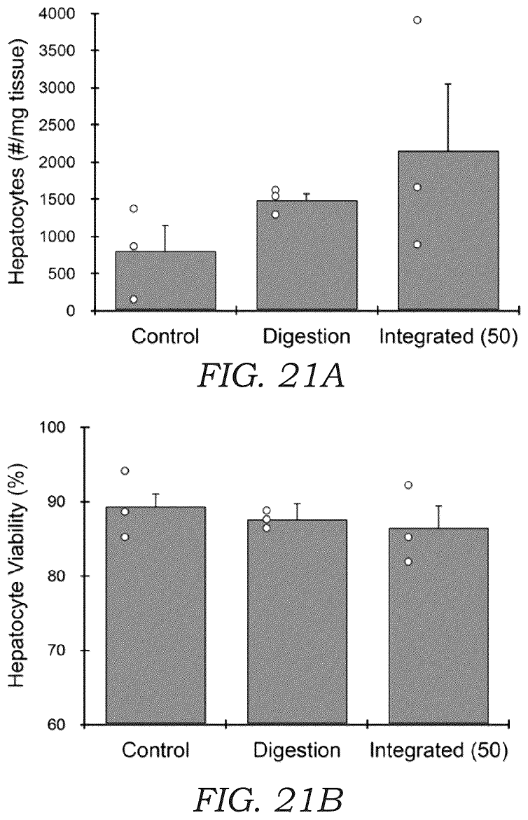

The liver plays a major role in drug metabolism and is frequently assessed for drug-induced toxicity. In fact, liver damage is one of the leading causes of post-approval drug withdrawal. Thus, in vitro screening of drugs against primary liver tissue is a critical component of preclinical testing. Increasingly, organ-on-a-chip systems are being employed to better maintain hepatocyte functionality and activity in culture settings and to enable personalized testing on patient-derived primary cells. While liver is softer and generally easier to dissociate, hepatocytes are well known to be fragile, and thus liver presents a unique dissociation challenge. As such, it was hypothesized that shorter device processing times would be effective for liver. For these experiments, murine liver was minced into 1 mm³ pieces and hepatocytes were detected based on ASGPR1 expression. Liver was first processed using the minced digestion device 12 for either 15 or 60 min. After 15 min, hepatocyte recovery was ˜4-fold higher for the device than the comparable control (FIG. 8A). Continued digestion of the control increased hepatocyte numbers further. Counterintuitively, continued processing in the digestion device 12 diminished hepatocyte yield by approximately half. It is believed that this finding was caused by the combination of two factors: softer liver tissue is effectively broken down at earlier time points and fragile hepatocytes are more sensitive to damage from recirculation. A single pass through the integrated dissociation/filtration device 40 was also tested, and found that hepatocyte recovery decreased. This was likely due to the large size of hepatocytes (˜30 μm), which caused them to be retained or damaged by the 15 μm membrane 64. It also appears that damage may have been additive, as viability dropped to 45% after 60 min digestion device treatment and passing through the integrated device 40, while all other conditions were ˜80% (FIG. 8B). Removing the 15 μm filter 64 from the integrated dissociation/filter device 40 increased hepatocytes by 30% relative to the digestion device alone 12, and by nearly 3-fold relative to the control, while maintaining viability (see FIGS. 21A-21B).

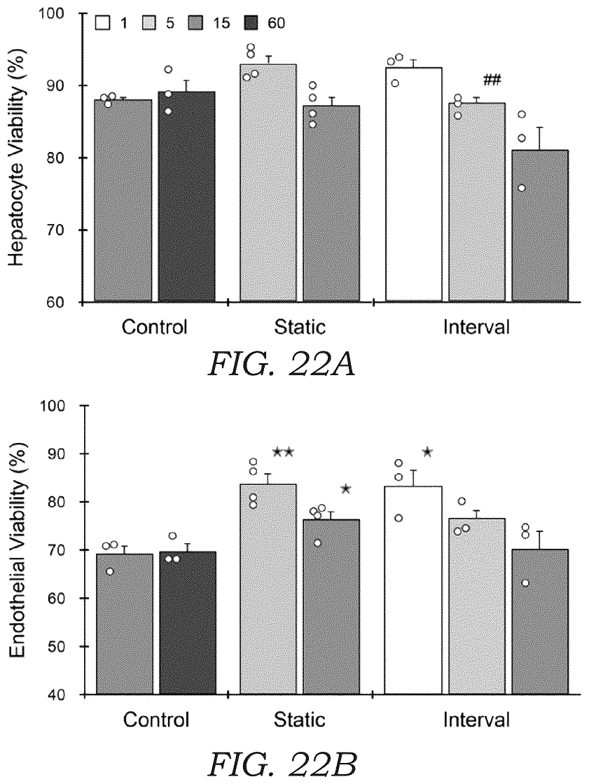

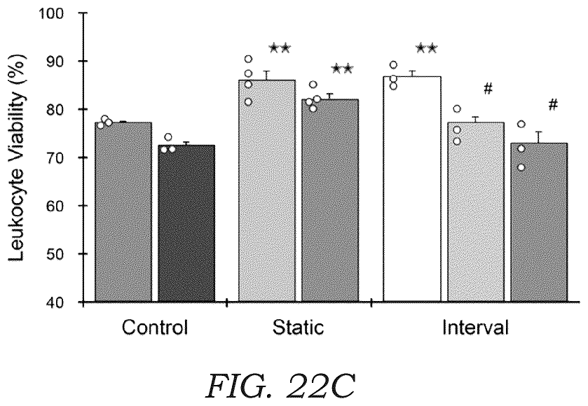

Based on the initial optimization studies, it was concluded that the microfluidic system 10 should utilize short processing times, and use the modified dissociation/filter device 40 with only the 50 μm filter 62. After 5 min digestion device processing, ˜700 hepatocytes were recovered/mg liver tissue (FIG. 8C). This was 4-fold higher than the 15 min control and just slightly less than the 60 min control (˜1000 hepatocytes/mg). Increasing digestion device 12 processing time to 15 min enhanced hepatocyte recovery by 40%, to the same level as the 60 min control. The most striking results were observed under the interval format. After only 1 min, ˜700 hepatocytes/mg tissue were recovered. Adding the 5- and 15-min intervals resulted in ˜2400 hepatocytes/mg, for a ˜2.5-fold enhancement relative to both the 60 min control and 15 min static conditions. Hepatocyte viability remained at 90% for controls and most device conditions (see FIG. 22A). Similar trends were observed for endothelial cells (FIG. 8D) and leukocytes (FIG. 8E), including significant recovery from the 1 min interval and enhanced overall cell numbers using the interval format. For endothelial cells, interval operation was ˜1.5-fold higher than the 60 min control and 15 min static device cases. For leukocytes, static device operation produced >2.5-fold more cells than the 60 min control, and interval operation further enhanced recovery to ˜3.5-fold. Given the strong performance of the device platform with leukocytes and their relative abundance in liver compared to kidney and tumor, cell suspensions were enriched for leukocytes in comparison to the 60 min control. This was particularly true for the static time points and the 1 min interval. Interestingly, the three interval conditions contained very different representations of hepatocytes and leukocytes, suggesting that the choice of elution time could serve as a means to crudely select for one population over the other, if that was so desired. Batch-to-batch reproducibility was highest for microfluidic processing using intervals for all but endothelial cells as seen in Table 7 below. Viability for endothelial cells and leukocytes remained similar to or greater than controls (see FIGS. 22B and 22C).

TABLE 7

| Condition | Hepatocytes | Endothelial Cells | Leukocytes |
|---|---|---|---|
| Control 15 m | 27.6 | 5.5 | 22.7 |
| Control 60 m | 26.7 | 8.6 | 15.8 |
| Static 5 m | 13.8 | 11.5 | 16.7 |
| Static 15 m | 26.5 | 11.7 | 8.6 |
| Interval 1 m | 20.6 | 11.3 | 3.7 |
| Interval 5 m | 14.2 | 8.4 | 7.7 |
| Interval 15 m | 14.5 | 11.5 | 4.9 |

Table 7 shows coefficient of variation values for liver samples at different processing conditions.

Taken together, the performance of the microfluidic system 10 with liver was quite unique relative to kidney and tumor. It is believed that this caused by the fact that fluid shear forces are needed to break down tissue, but can also damage some cell types that have already been liberated. All tissues require proper balancing of these effects. For softer tissues like liver, the balance must be shifted away from breakdown and towards preservation, particularly for sensitive hepatocytes, which can be accomplished using interval recovery. Endothelial cells and leukocytes also exhibited some sensitivity to over-processing, although to a lesser degree. It is unclear whether this finding can be generalized to other tissues, including kidney and tumor. Liver sinusoidal endothelial cells are highly specialized, with abundant fenestrae and no underlying basement membrane. These properties could also make sinusoidal endothelial cells particularly sensitive to damage. For leukocytes, there was no distinguishing between those that originated within the liver, which would predominantly be Kupffer cells, from those that came from blood, which may be less sensitive to shear. Future studies directly assessing Kupffer cells, as well as hepatic stellate cells, would be of high interest, particularly to make progress towards complex liver models that utilize multiple cell types.

Isolation of Cardiomyocytes from Murine Heart

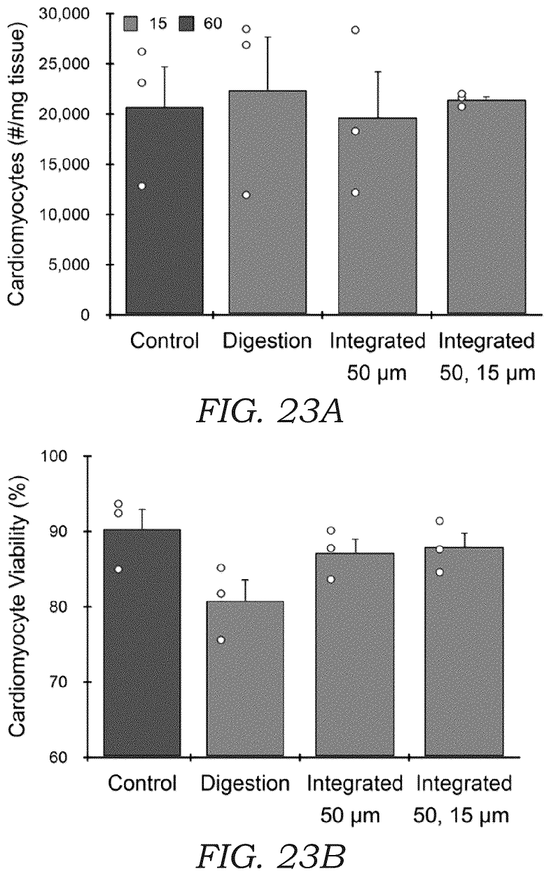

Heart failure is another leading cause of drug withdrawal from the market, combining with liver failure to account for ˜70% of withdrawals. Thus, there is robust interest in developing heart-on-chip technologies using primary cardiomyocytes for preclinical drug screening. Cardiomyocytes have been shown to be highly sensitive to mechanical and enzymatic dissociation techniques. In addition, they are disproportionately long in one direction, on the order of 100 μm and more. For these experiments, murine heart was minced into ˜1 mm³ pieces and cardiomyocytes were detected based on Troponin T expression. Since Troponin T is an intracellular marker, a fixable viability dye was used, Zombie Violet, in place of 7-AAD. Given potential concerns about cardiomyocyte size and shape, the effect of filter pore size in the integrated dissociation/filtration device 40 was tested. After 15 min processing with the minced digestion device 12, sample was passed through the original integrated dissociation/filter device 40 with both 50 and 15 μm pore size membranes 62, 64 or the modified version with only the 50 μm membrane 62. Cell numbers and viability were similar for all conditions (see FIGS. 23A-23B), and the original version with both membranes 62, 64 was selected for heart tissue.

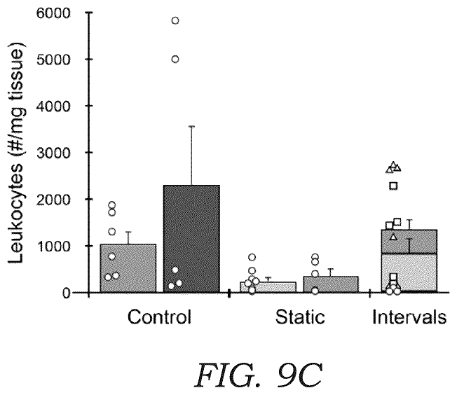

Next, the full microfluidic system 10 was evaluated at different digestion times. Shorter processing times were used due to the potential sensitivity of cardiomyocytes. After 5 min treatment with the digestion device 12, ˜2000 cardiomyocytes were recovered per mg heart tissue (FIG. 9A). This was lower than both the 15- and 60-min controls, by ˜half and one-third, respectively. Increasing digestion device processing to 15 min increased recovery to ˜12,000 cells/mg, which was ˜2-fold higher than the 60 min control. As with kidney, the interval format further increased cardiomyocyte recovery to ˜18,000 cells/mg. Endothelial cell (FIG. 9B) and leukocyte (FIG. 9C) yields from the microfluidic system 10 were significantly lower than the 60 min control. The interval format did improve recovery for both cases, but the 60 min control remained higher by ˜2-fold for endothelial cells and ˜1.5-fold for leukocytes. Based on this differential recovery, device platform 10 processing resulted in significant enrichment of cardiomyocytes. Batch-to-batch reproducibility was highest for microfluidic processing using intervals (see Table 8 below).

TABLE 8

| Condition | Cardiomyocytes | Endothelial Cells | Leukocytes |
|---|---|---|---|
| Control 15 m | 13.3 | 35.7 | 26.1 |
| Control 60 m | 14.1 | 26.8 | 54.5 |
| Static 5 m | 15.7 | 22.8 | 33.6 |
| Static 15 m | 36.2 | 30.8 | 40.2 |
| Interval 1 m | 28.9 | 27.4 | 28.4 |
| Interval 5 m | 12.7 | 21.5 | 40.1 |
| Interval 15 m | 7.1 | 22.5 | 34.5 |

Table 8 shows coefficient of variation values for heart samples at different processing conditions.

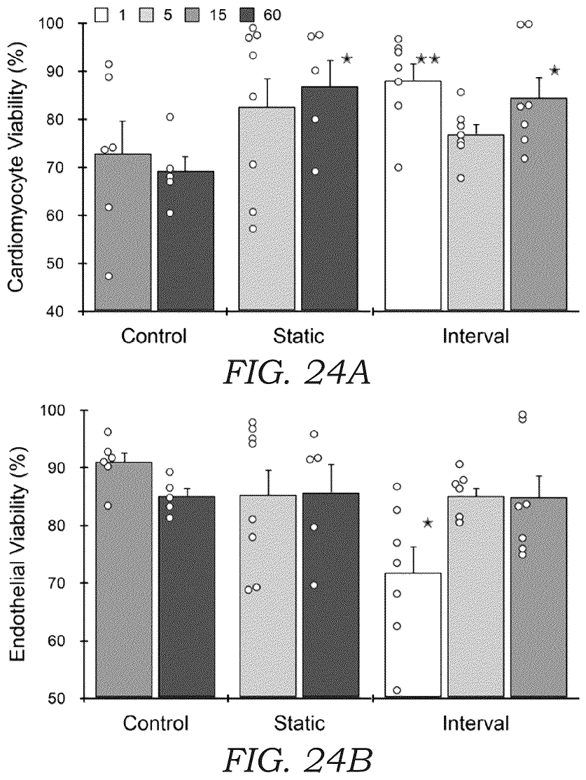

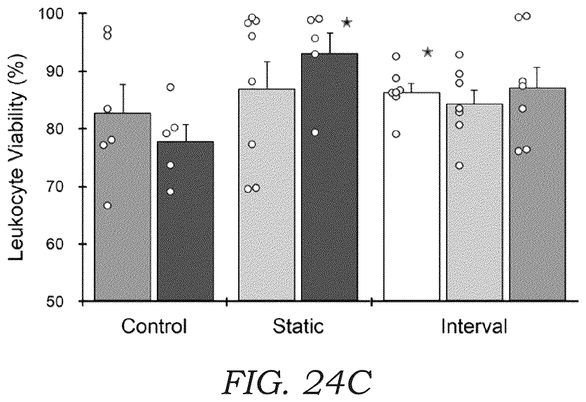

Viabilities for all three cells types were similar to controls (see FIGS. 24A-24C). Considering results for all tissues in a comprehensive manner, heart likely lies in between the kidney/tumor and liver extremes. The tissue is still challenging to break down, which is why recovery was low at the early time points. Digestion was likely to be particularly ineffective on its own for cardiomyocytes due to strong intracellular connections formed by desmosomes and adherins junctions, while the microfluidic system 10 provided the shear stresses necessary to break these connections and separate cardiomyocytes. However, the sensitivity of cardiomyocytes to mechanical damage is a confounding factor, which makes longer digestion times unlikely to improve results. Endothelial cells can arise from both vessels and the endocardium that lines the walls of the atrial and ventricular chambers. It is believed that endocardium was liberated effectively by digestion alone since the chambers can be readily accessed by collagenase. As seen for kidney and tumor, however, blood vessels require longer time for effective release of endothelium, even with the microfluidic system 10. This suggests that the results were dominated by endocardium, and that damage was the predominant reason for reduced recovery. The fact that interval recovery improved results for all cell types assessed in both heart and liver indicates that this mode is critical for optimal performance. In fact, temporal resolution should likely be increased, or ideally, be continuous, to prevent cell damage. Nevertheless, the microfluidic platform as currently configured and operated in this study consistently improved the recovery of single cells from diverse tissue types based on increased total cell yield, decreased processing time, and in some cases, both.

A novel microfluidic system 10 is disclosed that includes a digestion device 12 that facilitates loading and processing of minced specimens, as well as a newly integrated dissociation/filter device 40 that completes the dissociation workflow so that the single cell suspension is immediately ready for downstream analysis or alternative application. The new minced digestion device 12 accelerated tissue break down and produced significantly more single cells than traditional methods, while the integrated dissociation/filter device 40 increased yield further, all without affecting viability. This was determined for a diverse array of tissue types that exhibited a wide range of properties, as well as two different single cell analysis methods, flow cytometry and scRNA-seq. A novel processing scheme was used, including interval operation, which allowed the extraction of single cells at different time points during microfluidic digestion. It was found that for tissues that were physically tougher and more robust, such as kidney and tumor, microfluidic processing produced similar cell numbers in dramatically less time (15 vs 60 min), and approximately 2.5-fold more single cells in total. scRNA-seq further confirmed that endothelial cells, fibroblasts, and basal epithelial cells were highly enriched by the microfluidic system 19, with each increasing by 4- to 10-fold. Additionally, it was found that shorter digestion times were associated with lower stress responses for some cell types, but otherwise microfluidic processing did not add to the stress response in any case. These results clearly confirm that the microfluidic tissue system 10 holds exciting potential to advance scRNA-seq studies by reducing cell subtype-biasing, processing time, and/or stress response. For tissues that were softer and may contain sensitive cell types, like liver and heart, it was found that processing times could be dramatically reduced and that interval operation was critical to avoid cell damage and maximize recovery. These results will advance goals in tissue engineering and regenerative medicine, and could be particularly exciting for patient-derived organ-on-a-chip models for pharmacological studies. By focusing on minced specimens, the microfluidic tissue processing system 10 can readily be incorporated into the dissociation workflows for most, if not all, organs and tissues. Minimizing tissue pre-processing would be advantageous, and will be pursued in future work. Another future goal will be to decrease interval recovery time points to further explore protection of fragile cells, intentional enrichment of certain cell subtypes, and lowering of stress responses. Ideally, one would integrate a cell separation strategy that would make it possible to elute single cells from the platform as soon as they are generated. The microfluidic system 10 may be used for diverse tissue properties and cell subtypes. In addition, alternative proteolytic enzymes such as cold-active proteases may be used. Finally, microfluidic cell sorting and detection capabilities may be incorporated into the system 10 to create fully integrated and point-of-care technologies for cell-based diagnostics and drug testing, with a focus on human tissues for clinical applications.

Materials & Methods

Device Fabrication. Microfluidic minced digestion devices 12 and integrated dissociation/filter devices 40 were fabricated by ALine, Inc. (Rancho Dominguez, CA). Briefly, fluidic channels, vias, and openings for membranes, luer ports, and hose barbs were etched into PMMA polyethylene terephthalate (PET) layers using a CO₂ laser. Nylon mesh membranes (filters 62, 64) were purchased from Amazon Small Parts (15, and 50 μm pore sizes; Seattle, WA) as large sheets and were cut to size using the CO₂ laser. Device layers and other components (hose barbs, nylon mesh membranes) were then assembled, bonded using adhesive, and pressure laminated to form monolithic devices.

Murine Tissue Models. Kidney, liver, and heart were harvested from freshly sacrificed BALB/c or C57B/6 mice (Jackson Laboratory, Bar Harbor, ME) that were determined to be waste from a research study approved by the University of California, Irvine's Institutional Animal Care and Use Committee (courtesy of Dr. Angela G. Fleischman). Mammary tumors were harvested from freshly sacrificed MMTV-PyMT mice (Jackson Laboratory, Bar Harbor, ME). For kidneys, a scalpel was used to prepare ˜1 cm long×˜1 mm diameter strips of tissue, each containing histologically similar portions of the medulla and cortex. Tissue strips were then further minced with a scalpel to ˜1 mm³ pieces. Liver, mammary tumor, and heart were uniformly minced with a scalpel to ˜1 mm³ pieces. Minced tissue samples were then weighed and either processed with the devices as described below. Controls were placed within microcentrifuge tubes, digested at 37° C. in a shaking incubator under gentle agitation for 15, 30, or 60 min, and mechanically disaggregated by repeated pipetting and vortexing. 0.25% collagenase type I (Stemcell Technologies, Vancouver, BC) was used for both control and device-processed conditions. Finally, cell suspensions were treated with 100 Units of DNase I (Roche, Indianapolis, IN) for 10 min at 37° C. and washed by centrifugation into PBS+.

Minced Digestion Device Operation. Minced digestion devices 12 were prepared by affixing 0.05″ ID tubing 24 (Saint-Gobain, Malvern, PA) to the device inlet 14 and outlet 16 hose barbs, which was then connected to an Ismatec peristaltic pump 18 (Cole-Parmer, Werheim, Germany) with 2.62 mm ID tubing 24 (Saint-Gobain, Malvern, PA). Prior to experiments, devices 12, 40 and tubing 24 were incubated with SuperBlock (PBS) blocking buffer (Thermo Fisher Scientific, Waltham, MA) at room temperature for 15 min to reduce non-specific binding of cells to channel walls and washed with PBS+. Minced pieces of tissue were loaded into the device tissue chamber 20 through the luer inlet port 30. Devices 12 and tubing 24 were then primed with 0.25% collagenase type I solution (StemCell Technologies, Vancouver, BC), and the luer port 30 was closed off using a stopcock. The experimental setup consisting of the device 12, tubing 24, and peristaltic pump 18 were then placed inside a 37° C. incubator to maintain optimal enzymatic activity. The collagenase solution was recirculated through the device 12 and tubing 24 using the peristaltic pump 18 at a flow rate of 10 or 20 mL/min for a specified time.

Quantification of DNA Recovered from Cell Suspensions. Purified genomic DNA (gDNA) content of digested kidney tissue suspensions were assessed using a Nanodrop ND-1000 (Thermo Fisher, Waltham, MA) following isolation using a QIAamp DNA Mini Kit (Qiagen, Germantown, MD) according to manufacturer instructions. gDNA for device processed samples represents the cellular contents eluted from the device after operation, while gDNA for control samples represent the total amount of gDNA present in these samples.

Integrated Dissociation/Filter Device Operation. Following processing with the minced digestion device 12, tubing 24 was connected from the outlet 16 of the minced digestion device 12 to the inlet 46 of the integrated dissociation and filtration device 40 as seen in FIGS. 1A and 1F. If recirculation was intended, tubing 24 was connected from the cross-flow outlet to the peristaltic pump 18, while the outlet 48 of the integrated device 40 was closed off with a stopcock. Fluid was then pumped through the dissociation/filtration device 40 at 10 mL/min flow rate with pump 44. For final collection of the sample, or if only 1 pass through the dissociation component was utilized, the cross-flow outlet was closed off with a stopcock valve, and sample was pumped through at 10 mL/min and collected from the effluent outlet 50. Following all experiments, devices 12, 40 were washed with 2 mL PBS+ to flush out and collect any remaining cells. For time interval recovery, each PBS+ wash was followed by repriming of the device 12, 40 and tubing with collagenase solution, and the outlet 16 of the minced digestion device 12 was reconnected to the peristaltic pump 18 for continued recirculation until the next collection period.

Analysis of Cell Suspensions using Flow Cytometry. Cell suspensions were analyzed using tissue specific flow cytometry panels shown in Table 1. For initial studies with kidney, cell suspensions were stained concurrently with 5 μg/mL anti-mouse CD45-AF488 (clone 30-F11, BioLegend, San Diego, CA), 7 μg/mL EpCAM-PE (clone G8.8, BioLegend, San Diego, CA), and 5 μg/mL TER119-AF647 (clone TER-119, BioLegend, San Diego, CA) monoclonal antibodies for 30 minutes. Samples were then washed twice with PBS+ by centrifugation, stained with 3.33 μg/mL 7-AAD viability dye (BD Biosciences, San Jose, CA) on ice for at least 10 minutes, and analyzed on a Novocyte 3000 Flow Cytometer (ACEA Biosciences, San Diego, CA). Flow cytometry data was compensated using single stained cell samples or compensation beads (Invitrogen, Waltham, MA). Gates encompassing the positive and negative subpopulations within each compensation sample were used calculate a compensation matrix in FlowJo (FlowJo, Ashland, OR). A sequential gating scheme (see FIG. 25) was used to identify live and dead single epithelial cells, leukocytes, and red blood cells. Signal positivity was determined using appropriate Fluorescence Minus One (FMO) controls. Final studies with kidney, tumor, and liver used BV510 with CD45 (12.5 μg/mL, BioLegend, San Diego, CA) and also incorporated 8 μg/mL CD31-AF488 for endothelial cells. Liver demonstrations also replaced EpCAM-PE with 10 μg/mL ASGPR1-PE (clone 8D7, Santa Cruz Biotechnology, Dallas, TX) for hepatocytes. Heart demonstrations used 1:1000 dilution of Zombie Violet (Biolegend, San Diego, CA) instead of 7-AAD for viability, and replaced EpCAM-PE with 0.15 μg/mL Troponin T-PE (clone REA400, Milentyi Biotec, San Diego, CA) for cardiomyocytes.

Single Cell RNA Sequencing. These studies used 12-week old mice (male, C57BL/6 for kidney; female, MMTV-PyMT for mammary tumor, both from Jackson Laboratory, Bar Harbor, ME), which were euthanized by CO₂ inhalation. Kidneys and mammary tumor were dissected, minced into ˜1 mm³ pieces, and prepared as described for the microfluidic system 10 (15- and 60-min digestion device 12 intervals, single pass-through integrated dissociation/filter device 40) or control (60 min digest) using 0.25% type I collagenase. Recovered cells were centrifuged (400×g, 5 min), treated with 100 Units of DNase I for 5 min at 37° C., and washed by centrifugation into PBS+. Samples were then incubated with RBC lysis buffer for 5 min on ice, centrifuged, and resuspended in PBS+. Cells were stained with SytoxBlue (Life Technologies, Carlsbad, CA, USA) prior to FACS (FACSAria Fusion, BD Biosciences, Franklin Lakes, NJ) to remove dead cells and ambient RNA. Sorted live single cells (SytoxBlue-neg) were centrifuged and resuspended at a concentration of 1000 cells/μL in PBS with 0.04% BSA. The 10× Chromium system (10× Genomics, Pleasanton, CA) was then used for droplet-enabled scRNA-seq. Oil, cells, reagents, and beads were loaded onto an eight-channel microfluidic chip. Lanes were loaded with ˜17,000 cells from each of the samples, determined using an automated cell counter (Countess II, Invitrogen, Carlsbad, CA). Library generation for 10× Genomics Single Cell Expression v3 chemistry was then performed according to manufacturer's instructions. An Illumina NovaSeq 6000 platform (Illumina, San Diego, CA) was used to sequence the samples at a depth of ˜60,000 reads/cell for kidney and ˜45,000 reads/cell for mammary tumor. Sequencing fastq files were aligned using 10× Genomics Cell Ranger software (version 3.1.0) to an indexed mm10 reference genome. Cell Ranger Aggr was used to normalize the mapped reads for cells across the libraries for each data set. Genes that were not detected in at least 3 cells were discarded from further analysis. Cells with low (<200) or high (>3000 for kidney; >4000 for mammary tumor) unique genes expressed were also discarded, as these potentially represent low quality or doublet cells, respectively. Cells with high mitochondrial gene percentages were also discarded (>50% for kidney and >25% for mammary tumor), as these can also represent low quality or dying cells. The Seurat pipeline was used for cluster identification, principal component analysis (PCA) was performed using genes that are highly variable, density clustering was performed to identify groups, and Uniform Manifold Approximation and Projection (UMAP) plots were used to visualize the groupings. For kidney, cell clusters were annotated using two approaches. First, top differential genes in each cluster were examined to determine the cell type of the cluster based on expression of known marker genes (e.g., Kap, Napsa, and Slc27a2 for S2-S3 proximal tubules, Gpx3 for S1 proximal tubules, Emcn for endothelial cells, Slc12a1 for loop of Henle, Slc12a3 for distal convoluted tubule, etc. Second, since a well-established atlas of murine kidney was available, a cell scoring method was used to compare marker gene signatures from each of the clusters to published datasets to confirm cluster annotations (see FIGS. 14A-14B). For tumor, cell clusters were annotated by examining top differential genes in each cluster to determine cell type based on expression of known marker genes (e.g., EpCAM for epithelial cells). Cellular stress responses were assessed using a previously developed scoring method to compare stress response gene expression from each cluster to a previously published dataset of known stress response genes.

Cell Aggregate Studies. MCF-7 human breast cancer cells were obtained from ATCC (Manassas, VA) and cultured as recommended. Prior to experiments, confluent monolayers were briefly digested for 5 min with trypsin-EDTA, which released cells with a substantial number of aggregates. Cell suspensions were prepared for experiments by centrifugation and resuspension in PBS containing 1% BSA (PBS+). MCF-7 cells were then recirculated through the peristaltic pump system alone, or the system with a digestion device 12 or integrated dissociation/filter device 40 attached using methods described herein. For this initial study, flow was recirculated only through the dissociation portion of the integrated device 40 but not passed through the nylon filters 62, 64 of the filtration component for final sample collection in order to avoid confounding effects. To achieve this, the effluent outlet 50 of the integrated device 40 was closed off during pump operation using a stopcock. For all three tests, 5, 10, or 20 mL/min flow rates were used, and recirculation times of 0.5, 1, 4, and 10 min. Following experiments, devices 12, 40 and tubing 24 were washed with 2 mL PBS+ to flush out and collect any remaining cells. Cell counts and viability were obtained both before and after recirculation using a Moxi Flow cytometer with type MF-S cassettes (Orfo, Hailey, ID) and propidium iodide staining.

Flow cytometry gating protocol. Cell suspensions obtained from digested murine kidney, mammary tumor, liver, and heart samples were stained with the fluorescent probes listed in Table 1 and analyzed using flow cytometry. Acquired data was compensated and assessed using a sequential gating scheme (FIG. 25). Gate 1 was based on FSC-A vs. SSC-A, and was used to exclude debris near the origin. Gate 2 was used to select single cells based on FSC-A vs. FSC-H. Gate 3 distinguished leukocytes based on CD45-BV510 positive signal and TER119-AF647 negative signal, while red blood cells were identified based on TER119-AF647 positive signal and CD45-BV510 negative signal. Gate 4 was applied to the CD45⁽⁻⁾/TER119⁽⁻⁾ cell subset and used to identify epithelial cells in kidney and tumor samples based on positive EpCAM-PE signal, hepatocytes in liver samples based on positive ASGPR1-PE signal, and cardiomyocytes in heart samples based on positive Troponin T-PE signal. Gate 5 was applied to the EpCAM⁽⁻⁾ cell subset in kidney and tumor samples, the ASGPR1⁽⁻⁾ cell subset in liver, and the Troponin T⁽⁻⁾ cell subset in heart tissue to identify endothelial cells based on positive CD31-AF488 signal. Finally, gate 6 was used to identify live cells in epithelial, hepatocyte, cardiomyocyte, leukocyte, and endothelial cell subsets based on negative 7-AAD or Zombie Violet (heart) signal. Appropriate isotype controls were initially used to assess nonspecific background staining, and appropriate fluorescence minus one (FMO) controls were used to determine positive signal cut-offs and set gates. Control samples were left unstained.

Evaluation of Pump and Device Recirculation Using MCF-7 Cells

The effect of repeatedly recirculating cells through the peristaltic pump 18 and minced digestion device 12 using the MCF-7 human breast cancer cell line was investigated. This is a strongly cohesive cell type that retains a significant number of aggregates after routine cell culture, and thus requires more powerful dissociation methods. Prior to experiments, confluent monolayers were briefly digested with trypsin-EDTA, centrifuged, and resuspended in PBS containing 1% BSA (PBS+). Sample was then loaded into peristaltic tubing 24 that was either looped through the pump 18 or connected to a minced digestion device 12. Following recirculation for different periods of time at different flow rates, sample was collected for measurement of single cell number and viability (propidium iodide exclusion) using a Moxi flow cytometer. Results are presented in FIGS. 10A-10F, with cell numbers normalized to the control. It was found that recirculation through the pump 18 alone and the minced digestion device 12 were both associated with a modest decrease of ˜10 to 20% for all conditions tested, which was significant in many cases (FIGS. 10A and 10B). Cell viabilities were consistently ˜80%, similar to control. (FIGS. 10D and 10E). It should be noted that it was possible for cell number to increase due to aggregate dissociation or decrease due to cell destruction, and both of these factors should increase with hydrodynamic shear stress. Since total shear varied considerably across the conditions, both in terms of flow rate and processing time, the results suggest that the small decrease in cell number observed was associated with hold-up within the system or cell loss during transfer steps.

Next recirculation through the branching channel dissociation device 40 was tested. Previous work with this technology utilized a back-and-forth approach, which was achieved using a syringe pump. Here, the integrated dissociation/filter device 40 was used with flow recirculated only through the dissociation portion and not passed through the nylon filters 62, 64 so as to avoid confounding the results. Cell numbers obtained after recirculating at 5, 10, and 20 mL/min for 0.5, 1, 4. and 10 min are presented in FIG. 10C. No changes were observed at the 5 mL/min flow rate. At 10 mL/min, it was found that cell number increased modestly for short recirculation times, while longer recirculation enhanced single cell recovery by up to 2.5-fold. The 20 mL/min flow rate resulted in 2 to 4-fold increases for each time point. However, cell viability dropped precipitously for the conditions that provided the largest increases in single cell number (FIG. 10F). The modest increase in cell number observed at 10 mL/min for short recirculation times, on the order of ˜20%, is consistent with previous work using a syringe pump and a back-and-forth format. Moreover, the correlation between very large increases in single cell number and low viability was previously seen for the filter device when very small pore sizes (5 and 10 μm) were used. Based on these results, 10 mL/min was chosen as the optimal flow rate for the integrated dissociation/filter device 40, and focused on employing shorter processing times in order to increase single cell yields without compromising cell viability. The 10 mL/min is also the flow rate used with the dissociation/filtration device 40 with the filters 62, 64.

Platform Optimization Using Murine Kidney

The minced digestion device 12 and integrated dissociation/filter device 40 were separately optimized using murine kidney samples, and results for epithelial cells are presented in FIGS. 3A and 3C-3F. Single leukocytes were also quantified by flow cytometry via CD45, and results are presented in FIGS. 11A-11D. From the digestion device optimization study, it was found that leukocyte yield (FIG. 11A) and viability (FIG. 11B) followed similar trends as epithelial cells. Leukocytes increased with recirculation time in the digestion device 12, exceeding the control at 60 min, but by a more modest ˜30%. Moreover, both static and interval formats produced similar results. Leukocyte viability was higher with digestion device 12 processing for all but the 60 min interval. It was then investigated whether the integrated dissociation/filter device 40 could further enhance single cell yield following 15 min of digestion device processing. For leukocytes, recovery did not change for a single pass and decreased modestly with recirculation (FIG. 11C). Relative to the 15 min control, microfluidic device processing produced 7-fold more cells. Leukocyte viability displayed an upward trend with additional processing, but differences were not significant (FIG. 11D).

Single Cell Analysis of Murine Kidney

The full microfluidic system 10 or platform was evaluated using murine kidney samples, and results for epithelial cell, endothelial cell, and leukocyte numbers are presented in FIGS. 4A-4C. Single RBCs were also quantified by flow cytometry via TER119, and results are presented in FIG. 12. RBCs generally eluted at earlier timepoints for device processing, with nearly 50% recovered in the 1 min interval. A significant portion of these RBCs can likely be attributed to blood that was released during organ harvesting and mincing. However, RBCs did still increase with digestion time for controls, indicating that the digestion device 12 may rapidly wash out cells and blood from within undigested tissue. Cell viability was assessed by flow cytometry via 7-AAD dye, and results are presented in FIGS. 13A-13C. Epithelial viability was highest, at ˜95% for all control and device conditions (FIG. 13A). Endothelial (FIG. 13B) and leukocyte viabilities (FIG. 13C) ranged from ˜60% to 90%, with the 60 min control at ˜70% for both cases. Device processing resulted in higher viabilities for endothelial cells at all conditions except the 1 min interval, and leukocytes were elevated at the 15 min time points (static and interval).

scRNA-seq was performed on kidney samples, and identified seven cell clusters that are presented and analyzed in FIGS. 5A-5C. To confirm kidney cell cluster annotations, a cell scoring method was used by implementing the “AddModuleScore” function from Seurat to compare marker gene signatures from each of the main cell clusters (FIG. 14A) and subclusters (FIG. 14B) to established datasets. Each of the seven cell clusters are represented in the control and both microfluidic processing conditions. The LOH, DCT, CD, & MC cluster was evaluated by separating into the four different cell types. These correspond to the loop of Henle, distal convoluted tubule, collecting duct, and mesangial cells, which are each displayed in a UMAP diagram (FIG. 15A). The numbers obtained for each of these cells types are given in FIG. 15C, relative to the entire population. Each of these cell types were depleted in the 15 min platform interval, while the 60 min platform interval contained a proportional representation. A slight enrichment of LOH cells and depletion of CD cells in was found in the 60 min interval.

To facilitate correlations between scRNA-seq and flow cytometry results, gene expression of EpCAM, CD31, and CD45 was inspected. EpCAM was highly expressed predominantly in the main DCT, LOH, CD, & MC cluster (FIG. 16A), including each of the cell subsets (FIG. 16B). Proximal tubules were predominantly negative for EpCAM, possibly due to low basal expression and a potential secondary factor such as low protein turnover. CD45 was highly expressed in the macrophage, B lymphocyte, and T lymphocyte clusters (FIG. 16C), and CD31 was highly expressed in the endothelial cluster (FIG. 16D), as expected. In order to make quantitative comparisons, two assumptions were made. First, the cell numbers obtained by flow cytometry in FIGS. 4A-4C were determined and it was deduced that microfluidic processing produced approximately equal number of total cells in the 15 min interval and ˜50% more cells in the 60 min interval, relative to the 60 min control. Second, it was assumed that all proximal tubules, as well as all DCT, LOH, CD, and MC subtypes, are EpCAM positive. Based on these assumptions, the population percentages obtained for the 60 min device interval in FIG. 5B were weighted by 1.5 and added it to the 15 min values to estimate an aggregate value for the microfluidic platform. Results are presented in Table 2, which also includes normalization to the 60 min control and calculation of aggregate population percentages. Although these estimates require caveats, they do closely match flow cytometry results in FIGS. 4A-4C, with ˜2.5-fold more epithelial cells (proximal tubule, DCT, LOH, CD), ˜2- to 2.5-fold more leukocytes (macrophage, B and T lymphocytes), and ˜4-fold more endothelial cells produced with the microfluidic platform relative to the 60 min control. Moreover, aggregate population percentages for microfluidic processing were generally comparable to the 60 min control in FIG. 5B, with the exception that endothelial cells were enriched.

Processing and Single Cell Analysis of Murine Breast Tumor Tissue

The minced digestion device 12 and integrated dissociation/filter device 40 were separately optimized using a murine breast tumor model (transgenic MMTV-PyMT). Samples were processed using the minced digestion device 12 for 15, 30, or 60 min, and generated ˜2- to 2.5-fold more epithelial cells than controls at the same time points (FIG. 17A). Epithelial cell viability was lower for controls than for device conditions at all digestion times (FIG. 17B). Next, samples were passed through the integrated dissociation/filter device 40 following 15 min treatment with the digestion device 12. A single pass was found to be optimal in terms of epithelial cell yield (FIG. 17C) and viability (FIG. 17D), similar to kidney.

The full microfluidic system 10 was then evaluated, and results for epithelial cell, endothelial cell, and leukocyte numbers are presented in FIGS. 6A-6C. Cell viability was also assessed by flow cytometry via 7-AAD dye, and results are presented in FIGS. 18A-18C. Epithelial cell viabilities were ˜80% for all conditions except the 60 min control and 15 min device interval, which decreased to ˜70% (FIG. 18A). Endothelial cell viability was generally low at ˜60% (FIG. 18B). However, the 1 min device interval was higher at 75%, while the 60 min control and 15 min device interval were lower at 50% and 40%, respectively. Leukocyte viability remained ˜80% for all but the 60 min control, which was ˜60% (FIG. 18C).

scRNA-seq was also performed and six cell clusters were identified that are presented and analyzed in FIGS. 7A-7C. Epithelial cells were the predominant cluster, three sub-clusters were further identified that corresponded to luminal, basal, and proliferating luminal cells (FIG. 19A). These sub-clusters were associated with expression of Krt14, Krt18, and Mki67 genes (FIG. 19B). Population percentages, relative to the full population, are presented in FIG. 19C. The luminal subtype was enriched in the 15 min interval, the basal subtype was enriched in the 60 min interval, and the proliferating luminal was under-represented at both time points.

Lastly, scRNA-seq results were correlated to flow cytometry in a similar manner as kidney. EpCAM was now well-correlated with the main epithelial cluster (FIG. 20A), as well as each sub-cluster (FIG. 20B). CD45 was highly expressed in macrophage, T lymphocyte, and granulocyte clusters (FIG. 20C), while CD31 was highly expressed in the endothelial cluster (FIG. 20D). Microfluidic system 10 results were then aggregated using the same approach described for kidney, with 60 min interval results weighted by 1.5 and added to 15 min interval values, and results are presented in Table 3. These estimates again matched flow cytometry results for each cell population (FIGS. 6A-6C), with ˜2-fold more epithelial cells and ˜4-fold more endothelial cells produced with the microfluidic system 10 relative to the 60 min control. Leukocyte values relative to the control were 3-fold higher for macrophages, 2.5-fold higher for T lymphocytes, and 20% lower for granulocytes. Notably, substantial increases for fibroblasts (>10-fold) and basal epithelial cells (>6-fold) were found with microfluidic processing. Aggregate population percentages for the microfluidic platform were generally comparable to the 60 min control in FIG. 7B, but with significant enrichment of macrophages, endothelial cells, and fibroblasts.

Isolation of Hepatocytes from Murine Liver

The minced digestion device 12 and integrated dissociation/filter device 40 were tested separately using murine liver, and found that the integrated device 40 decreased hepatocyte yield (FIG. 8A) and viability (FIG. 8B). It was hypothesized that the second filter 64, with a pore size of 15 μm, was too small for large and fragile hepatocytes. Therefore, a modified version of the integrated dissociation/filter device 40 was created that omitted the second filter 64. After processing liver for 15 min with the minced digestion device 12, the cell suspension was passed through the modified dissociation/filter device 40 one time, which increased hepatocytes by 30% relative to the digestion device 12 alone and by nearly 3-fold relative to the control (FIG. 21A). Hepatocyte viability was preserved, remaining >85% for all conditions (FIG. 21B).

The full microfluidic system 10 (with modified single filter 62 configuration) was then evaluated, and results for hepatocyte, endothelial cell, and leukocyte numbers are presented in FIGS. 8A-8E. Cell viability was assessed by flow cytometry via 7-AAD dye, and results are presented in FIGS. 22A-22C. Hepatocyte viability remained at ˜90% for most conditions tested (FIG. 22A). A small increase was observed for static or interval processing conditions, but values were not significantly different than controls. Endothelial cell (FIG. 22B) and leukocyte (FIG. 22C) viabilities followed similar trends seen in hepatocytes, and were generally between ˜70% and 85%.

Isolation of Cardiomyocytes from Murine Heart

The minced digestion device was tested, with and without the integrated dissociation/filter device 40 using murine heart. This included both the original integrated device 40 and the modified device 40 without the 15 μm filter 64 that was created for liver. It was found that after processing heart tissue for 15 min, cardiomyocyte numbers and viability were unchanged for each case (FIGS. 23A-23B). As a result, the standard version of the integrated dissociation/filter device 40 was selected to be used with both 50 and 15 μm filters 62, 64 for heart tissue.

The full microfluidic system 10 was then evaluated, and results for cardiomyocyte, endothelial cell, and leukocyte numbers are presented in FIGS. 9A-9C. Cell viability was assessed by flow cytometry via Zombie Violet dye, and results are presented in FIGS. 24A-24C. Cardiomyocyte viability was ˜70% for controls, while values for device processing were all at ˜75-85% (FIG. 24A). Viabilities for endothelial cells (FIG. 24B) and leukocytes (FIG. 24C) were generally >80% for all device and control conditions.

Statistics. Data are represented as the mean±standard error. Error bars represent the standard error from at least three independent experiments. P-values were calculated from at least three independent experiments using students t-test.

While embodiments of the present invention have been shown and described, various modifications may be made without departing from the scope of the present invention. The invention, therefore, should not be limited, except to the following claims, and their equivalents.

What is claimed is:

1. A microfluidic system for processing a tissue sample comprising:
a microfluidic digestion device comprising an inlet and an outlet and a flow path defined between the inlet and the outlet, the flow path comprising a tissue chamber configured to hold the tissue sample and a plurality of upstream fluidic channels communicating with the tissue chamber on the inlet side of the flow path and a plurality of downstream fluidic channels communicating with the tissue chamber on the outlet side of the flow path;
a first pump configured to pump a buffer-containing fluid and/or an enzyme-containing fluid into the inlet of the microfluidic digestion device;
a microfluidic dissociation/filter device comprising an inlet, a first outlet, a second outlet, and a flow path defined between the inlet and the outlet, the flow path comprising a plurality of furcating dissociation channels having a plurality of expansion and constriction regions disposed along a length thereof, wherein one or more filters are disposed in the flow path downstream of the plurality of furcating dissociation channels;
a second pump configured to pump a buffer-containing or other fluid into the inlet of the microfluidic dissociation/filter device; and
wherein the outlet of the microfluidic digestion device is fluidically coupled to the inlet of the microfluidic dissociation/filter device.

2. The microfluidic system of claim 1, further comprising one or more valves interposed between the outlet of the microfluidic digestion device and the inlet of the microfluidic dissociation/filter device.

3. The microfluidic system of claim 1, wherein the first outlet comprises a valve or cap to close the same and wherein when the first outlet of the microfluidic dissociation/filter device is closed fluid causes fluid to flow through a flow path containing the one or more filters.

4. The microfluidic system of claim 1, wherein the second outlet comprises a valve or cap to close the same and wherein when the second outlet of the microfluidic dissociation/filter device is closed causes fluid to exit the microfluidic dissociation/filter device via the first outlet without passing through the one or more filters.

5. The microfluidic system of claim 1, wherein the one or more filters comprises a first filter having a pore size within the range of about 50-100 μm and a second filter having a pore size within the range of about 15-50 μm.

6. The microfluidic system of claim 1, wherein the number of upstream fluidic channels equals the number of downstream fluidic channels.

7. The microfluidic system of claim 6, wherein the upstream and downstream fluidic channels have a width within the range between about 250 μm and 750 μm.

8. The microfluidic system of claim 1, further comprising a port disposed on the microfluidic digestion device and in communication with the tissue chamber.

9. A method of using the microfluidic system of claim 1 comprising:
loading the tissue sample into the tissue chamber of the microfluidic digestion device;
pumping the buffer-containing fluid and/or an enzyme-containing fluid into the inlet of the microfluidic digestion device with the first pump;
transferring fluid containing processed tissue sample to the microfluidic dissociation/filter device;
pumping a buffer-containing or other fluid into the inlet of the microfluidic dissociation/filter device along with the processed tissue from the microfluidic digestion device with the second pump; and
collecting effluent from the second outlet of the microfluidic dissociation/filter device.

10. The method of claim 9, wherein pumping the buffer-containing fluid and/or an enzyme-containing fluid into the inlet of the microfluidic digestion device with the first pump comprises recirculating fluid into the microfluidic digestion device with the first pump.

11. The method of claim 9, wherein the enzyme-containing fluid comprises a fluid containing collagenase.

12. The method of claim 9, wherein the pumping the buffer-containing fluid and/or an enzyme-containing fluid into the inlet of the microfluidic digestion device is performed in intervals where effluent is removed from the microfluidic digestion device at the end of each interval and replacement enzyme-containing fluid is pumped into the microfluidic digestion device.

13. The method of claim 9, wherein total processing time in the microfluidic digestion device and the microfluidic dissociation/filter device is 1 minute or more.

14. The method of claim 9, wherein total processing time in the microfluidic digestion device and the microfluidic dissociation/filter device is 15 minutes or more.

15. The method of claim 9, wherein the processed tissue from the microfluidic digestion device is recirculated through the plurality of furcating dissociation channels in the microfluidic dissociation/filter device a plurality of times prior to exit from the second outlet.

16. The method of claim 9, wherein the microfluidic dissociation/filter device contains a single filter.

17. The method of claim 9, wherein the microfluidic dissociation/filter device contains a plurality of filters.
